(19) United States
(12) Patent Application Publication
(10) Pub. No.: US 2005/0233393 A1
(43) Pub. Date: Oct. 20, 2005

(54) Dynamic hormone index as a biomarker for disease

(76) Inventor: Katherine E. Wynne-Edwards, Kingston (CA)

Correspondence Address: LAHIVE & COCKFIELD, LLP., 28 STATE STREET, BOSTON, MA, 02109, US

(21) Appl. No.: 11/068,730

(22) Filed: Feb. 28, 2005

Related U.S. Application Data
(60) Provisional application No. 60/548,222, filed on Feb. 27, 2004.
(60) Provisional application No. 60/566,703, filed on Apr. 30, 2004.

(30) Foreign Application Priority Data
Feb. 27, 2004 (CA) 2,459,121

(51) Int. Cl. G01N 33/574; G01N 33/92

(52) U.S. Cl. 435/7.23; 436/71; 436/86

(57) ABSTRACT

The present invention relates, at least in part, to the novel discovery that hormone dynamics are predictive of an individual's risk of developing hormone-associated conditions such as cancer, metabolic syndrome X (MSX), obesity, stress, diabetes and symptoms of aging.

### RELATED APPLICATIONS

This application claims the benefit of U.S. Patent Application Ser. No. 60/548,222 filed Feb. 27, 2004, and of U.S. Patent Application Ser. No. 60/566,703 filed Apr. 30, 2004, the contents each of which are hereby incorporated by reference.

### BACKGROUND OF THE INVENTION

Many disease states, including cancer, cardiovascular disease and obesity have been linked to abnormal hormonal levels. For example, the levels of glucocorticoids, adrenocorticosteroids, and prostaglandins have been found to be altered in coronary heart disease and stress-related disorders. Many types of cancers, including breast, prostate, ovarian, cervical and colon cancer, have been linked to altered levels of steroids and androgens. In addition, steroid hormones, such as cortisol, as well as insulin levels, have been implicated in the development of obesity and related metabolic disorders.

Often, disorders that are associated with abnormal levels of hormones are also influenced by modifiable risk factors. For example, several modifiable risk factors have been linked to the risk of breast cancer in adult women, including physical inactivity (Friedenreich C M et al. Cancer 1998;83:600-10; Thune I et al. Med Sci Sports Exerc 2001;33:S530-S550), excess body weight (Calle E E et al. N Engl J Med 2003;348:1625-38; Huang Z P et al. J. Am. Med. Assoc. 1997;278:1407-11; Petrelli J M et al. Cancer Causes Control 2002;13:325-32), dietary animal fat consumption or intake (Cho E, et al. J Natl Cancer Inst 2003;95:1079-85), and excessive alcohol consumption (Hunter D J et al. Epidemiol Rev 1993;15:110-32). It is also plausible that adolescent lifestyle variables are risk factors for breast cancer (Frazier A L et al. Breast Cancer Research 2003;5:R59-R64; Okasha M et al. Breast Cancer Res Treat 2003;78:223-76; Romundstad P R, et al. Int J Cancer 2003; 105:400-3) and might provide important insights into the mechanisms linking lifestyle characteristics with biological risk (Lawlor D A et al. Br J Cancer 2003;89:81-7).

### SUMMARY OF THE INVENTION

During adolescence, hormones are active in breast development (Brisken C. J Mammary Gland Biol Neoplasia 2002;7:39-48), adult menstrual cycles become established, and breast tissue has a high intrinsic cell division rate (Russo J. et al. Breast Cancer Res Treat 1982;2:5-73). In addition, the adolescent years include reproductive, lifestyle and physical changes that are known or suspected risk factors for breast cancer. For example, adult height, body shape, lifetime patterns of physical activity, and dietary habits become established during these years or soon after (Apter D, Vihko R. J Clin Endocrinol Metab 1983;57:82-6; Bernstein L. J Mammary Gland Biol Neoplasia 2002;7:3-15; Li C I, et al. Epidemiology 1997;8:559-65). These anthropometric and lifestyle variables might affect breast cancer risk by altering individual exposure to sex steroids including estrogen, progesterone, testosterone, and DHEA (dehydroepiandrosterone) (Gustafsson J-A, Warner M. J Steroid Biochem Mol Biol 2000;74:245-8; Lillie E O, et al. Breast Cancer Research 2003;5:164-73; Berrino F, et al. J Natl Cancer Inst 1996;88:291-6; Cauley J A et al. Ann Intern Med 1999;130:270-7; Chlebowski R T, et al. J Am Med Assoc 2003;289:3243-53; Key T J, et al. J Natl Cancer Inst 2003;95:1218-26; McTieman A, et al. J Clin Oncol 2003;21:1961-6; Morris K T, et al. Surgery 2001;130:947-53; Reichman M E, et al. J Natl Cancer Inst 1993;85:722-7).

Thus, there still-exists a lack of complete understanding of the underlying biological mechanisms that link diet, lifestyle, and environmental factors to individual risk for disease. Trans-disciplinary integration, from biological mechanism to epidemiological risk, is urgently needed to understand the etiology of chronic disease, including cancer, obesity, inflammation and heart disease.

Accordingly, the present invention is based, at least in part, on the novel discovery that the analysis of hormone dynamics is predictive of an individual's risk of developing hormone-associated conditions such as, for example, cancer, metabolic syndrome X (MSX), obesity, stress, diabetes, symptoms of aging, cardiovascular disease and inflammation.

In one aspect, the invention provides a method for identifying a subject at risk of developing a hormone-associated condition using hormone dynamics. The method includes determining a dynamic hormone index of at least one hormone in the subject. For this method, an aberrant dynamic hormone index of at least one hormone in the subject when compared to the statistical distribution of a dynamic hormone index for a population is indicative that the subject is at risk of developing a hormone-associated condition. In one preferred embodiment, the dynamic hormone index of the subject is compared to the mean dynamic hormone index of a population.

In certain embodiments of this aspect of the invention, the dynamic hormone index is determined by measuring the level of at least one hormone in two or more biological samples obtained from the subject at two or more time points. In other embodiments, the dynamic hormone index is determined by measuring the level of two or more hormones in two or more biological samples obtained from the subject at two or more time points.

In another aspect, the invention provides a method of identifying a modifiable risk factor that has a statistically meaningful relationship with a hormone-associated condition. The method includes determining the dynamic hormone index of at least one hormone for individual subjects of a population, and determining the level of exposure of the individual subjects of the population to a suspected modifiable risk factor. In this method, a statistically meaningful relationship between an aberrant dynamic hormone index for a subset of the subjects in the population and the level of exposure of the subset to the risk factor is indicative that exposure to the suspected risk factor increases the likelihood of developing a hormone-associated condition.

In yet another aspect, the invention also provides a method of determining whether there is a statistically meaningful relationship between a known risk factor and an aberrant dynamic hormone index. The method includes determining the dynamic hormone index of at least one hormone for individual subjects of a population, and determining the level of exposure for individual subjects of the population to the risk factor. For this method, a statistically meaningful relationship between an aberrant dynamic hormone index of a subset of the subjects in the population and the level of exposure of the subset to the known risk factor is indicative that an exposure to the known risk factor increases the likelihood of developing an aberrant dynamic hormone index.

The invention also provides a method of reducing the risk of developing a hormone-associated condition in a subject identified as having an aberrant dynamic hormone index, by altering, e.g., reducing, the level of exposure of the subject to at least one modifiable risk factor associated with the hormone-associated disorder.

In a further aspect, the invention provides a method of monitoring the risk of developing a hormone-associated condition in a subject who has an aberrant dynamic hormone index. The method includes altering the exposure of the subject to at least one modifiable risk factor, and determining the dynamic hormone index of the subject after the exposure to the risk factor has been altered. In this method, a shift of the dynamic hormone index of the subject in a direction associated with lower health risk is indicative that the risk of the subject developing the hormone-associated condition is reduced. In certain embodiments, such a shift is toward the mean or median dynamic hormone index of the population.

In yet another aspect, the invention provides a method of identifying a course of treatment for a subject to reduce the subject's risk of developing a hormone-associated condition. The method includes monitoring the dynamic hormone index of the subject having an aberrant hormone index during a course of treatment. In this method, a shift of the dynamic hormone index of the subject toward the mean or median dynamic hormone index of the population during the course of treatment is indicative that the treatment reduces the subject's risk of developing the hormone-associated condition.

The invention also provides a method of determining whether a subject has an increased risk of developing one or more symptoms or adverse side effects of a hormone-associated condition. In certain embodiments, the method includes determining the relative level of at least one predictive hormone in two or more biological samples taken at two or more time points from the subject, and determining a dynamic hormone index for the subject. In other embodiments, the method includes determining the relative levels of at least two hormones in two or more biological samples taken at two or more time points from the subject. In this method, a dynamic hormone index for the subject that is outside of the mean or median dynamic hormone index of the population is indicative that the subject has an increased risk of developing one or more symptoms or adverse side effects of the hormone-associated condition.

The invention further provides a method of determining a subject's level of risk for developing a hormone-associated condition by determining the level of at least one hormone in two or more biological samples from the subject, and determining a dynamic hormone index for the subject. In this method, the extent to which the dynamic hormone index of the subject varies from the mean or median dynamic hormone index for a population is indicative of the subject's level of risk for developing the hormone-associated condition.

In still another aspect, the invention provides a method of determining a subject's level of risk for developing a hormone-associated condition by determining the level of at least two hormones in two or more biological samples taken from the subject at two or more time points, and determining a dynamic hormone index for the subject. In this method, the extent to which the dynamic hormone index of the subject varies from the mean or median dynamic hormone index for a population is indicative of the level of the subject's risk for developing the hormone-associated condition.

In still another aspect, the invention provides a kit for determining whether a subject has an increased risk of developing a hormone-associated condition. The kit includes means for determining the relative level of at least one predictive hormone in at least one sample from the subject, and instructions for identifying whether the individual is at risk of developing the hormone-associated condition. In some embodiments, the kit may also include means for determining the relative levels of two or more predictive hormones in at least one sample from the subject. In some embodiments, the kit may also include means for determining the relative level of one or more predictive hormone in at least in two or more samples from the subject taken at two or more time points. In preferred embodiments, the kit includes instructions for determining dynamic hormone index of at least one predictive hormone for which relative level(s) in sample(s) is determined.

### BRIEF DESCRIPTION OF THE DRAWINGS

FIG. 1 is a graph depicting hormone concentration and dynamic index for premenarcheal (open circle) and postmenarcheal (solid diamond) participants. Each hormone concentration represents the average of multiple samples for that individual. Panels for estradiol and progesterone use the left hand vertical axis (range 0-150%) whereas panels for testosterone and DHEA (dehydroepiandrosterone) use the right hand axis (range 0-80%). An inverse relationship for estradiol (R²=18.5%; P <0.001) is the result of assay measurement error at low concentrations (see Table 2). One individual with a measurement-imposed estradiol concentration of 1.0 pg/ml and dynamic index of zero was excluded. Estradiol dynamic index was not used in subsequent analyses.

FIG. 2a is a graph depicting the positive association between premenarcheal DHEA concentration and anthropometry (age-adjusted height; R²=46.1%, P=0.0005), and FIG. 2b is a graph depicting the positive association between premenarcheal DHEA concentration and lifestyle (dietary animal fat intake or consumption; R²=34.9%, P=0.004). For both figures, the DHEA concentration is adjusted for age plus the other variable. Values for R² and P represent the association in the model incorporating age, age-adjusted height, and dietary animal fat intake (%).

FIG. 3a is a graph depicting the positive association between premenarcheal progesterone dynamic index and physical inactivity (R²=50.1%; P=0.0002), and FIG. 3b is a graph depicting the positive association between testosterone dynamic index (standardized for pubic hair distribution) and dietary animal fat intake or consumption (R²=41.8%; P=0.002). Values for R² and P represent the association in the model incorporating significant variables.

FIG. 4 is a graph depicting the mean hormone concentration for individual hamsters on the high and low fat diets.

FIG. 5 is a graph depicting the dynamic hormone index for individual hamsters on the high and low fat diets.

FIG. 6A is a graph depicting log free testosterone concentration (pg/ml) for Group A, the group in which the exercise wheel was initially locked and then unlocked later to allow exercise. FIG. 6B is a graph depicting log free testosterone concentration (pg/ml) for Group B in which the exercise wheel was initially unlocked to allow exercise and then locked.

FIG. 7A is a graph depicting dynamic hormone index for free testosterone in individual hamsters from Group A. FIG. 7B is a graph depicting dynamic hormone index for free testosterone in individual hamsters from Group B.

FIG. 8 is a graph depicting the relationship between the change in log free testosterone concentration and the change in free testosterone dynamic index.

FIG. 9 is a bar graph depicting the dynamic index for free testosterone in control men (N=7), expectant/new fathers (N==12), and expectant/new mothers (N=8)

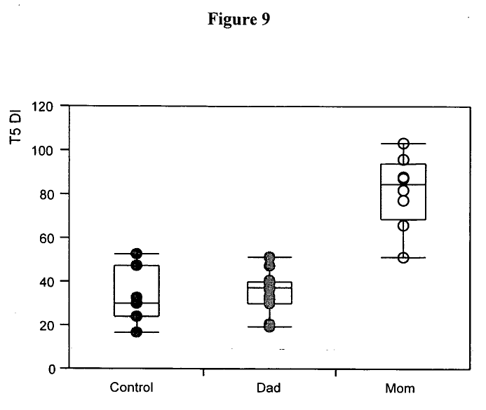

### DETAILED DESCRIPTION OF THE INVENTION

The invention pertains, at least in part, to methods of using an individual's dynamic hormone index (or an individual's hormone variability) as a biomarker for potential associations between anthropometric and lifestyle variables that have been previously linked to risk of hormone-associated conditions, such as breast cancer.

Individual variability measures have emerging clinical relevance in heart disease, chronic fatigue syndrome, and physical fitness (Javorka M et al. Clinical Physiology and Functional Imaging 2003;23:1-8; Karcz M et al. Int J Cardiol 2003;87:75-81; Singh J P et al. Am J Cardiol 2002;90:1290-1293; Yamamoto Y et al. Exp Biol Med 2003;228:167-174). However, a dynamic hormone index (i.e., within-individual variability in hormone concentration) has not previously been calculated or considered in the context of hormone-associated conditions, such as breast cancer. For example, two individuals with the same average hormone concentration might differ in their dynamic hormone index, with one ranging in variability from 25 to 175% of the average hormone concentration over time and the other ranging only from 90 to 110% of the average hormone concentration. Likewise, two individuals representing the low and the high end of the normal range might have exactly the same variability score. Physiologically, high and low variability over time will have different impacts on receptor binding and the activity of second messenger pathways. For example, one individual will see frequent doubling and/or halving of receptor binding, whereas another will not, resulting in different effects on the activity of second messenger pathways and/or gene activation in the respective individuals.

Accordingly, the invention provides a method for identifying a subject at risk of developing a hormone-associated condition using hormone dynamics. The method includes determining a dynamic hormone index of at least one hormone in the subject, and comparing it to the statistical distribution of a dynamic hormone index for a population. If the dynamic hormone index of the subject for at least one hormone is aberrant, the subject is at risk of developing a hormone-associated condition.

A “biomarker” is a biologically occurring characteristic or molecule that may be measured in a biological sample and evaluated as an indicator of normal biologic or pathogenic processes; of biological or pathogenic status; as an index of the risk or the progression of disease; as a measure of exposure to an environmental chemical or factor; and/or of pharmacological response to a therapeutic intervention. Examples of biomarkers include, but are not limited to, hormones, DNA, RNA, protein, peptide, carbohydrate, lipid and steroids.

The term “hormone” includes substances which affect the metabolism or behavior of cells which possess receptors for the particular hormone. As used herein, the term also includes precursors, fragments, metabolites, degradation products or other proxy indicators of the metabolically active form of the hormone which may be measured. Hormones may be naturally occurring in the subject; a natural product obtained from other biological organisms including, but not limited to plants (e.g., soybeans), recombinant organisms (e.g., bacteria or yeast producing a heterologous hormone) and a species other than the subject (e.g., a pregnant mare or cow); or chemically synthesized (e.g., contraceptives or anabolic steroids). Examples of hormones include, but are not limited to, glucocorticoids (e.g., cortisol, corticosterone, prednisone), mineralocorticoids (e.g., aldosterone, 1,1-deoxycorticosterone), androgens (e.g., testosterone, androstenedione, dihydrotestosterone, 11-ketotestosterone, dehydroepiandrosterone (DHEA)), estrogens (e.g., estradiol, estriol, estrone), progestagens (e.g., progesterone), thyroid hormone (e.g., T3, T4 and T5), melatonin, and leptin. Other examples of hormones include, but are not limited to parathyroid hormone, thyroxine, triiodothyronine, thyroid stimulating hormone, follicle-stimulating hormone, luteinizing hormone, growth hormone, met-enkephalin, leu-enkaphalin, cholecysotokinin, bombesin, gastric inhibitory peptide, motilin, vasoactive intestinal polypeptide, arginine-vasotocin, growth hormone-releasing hormone, melanotropin release inhibitory factor, melanotropin releasing factor, neurotensin, prolactin inhibiting hormone, prolactin releasing hormone, prolactin, mullerian regression hormone, substance P, relaxin, inhibin, vasopressin, lipotropin, melanocyte-stimulating hormone, adrenocorticotropic hormone, alpha melanocyte-stimulating hormone, antidiuretic hormone, thyrotropin-releasing hormone, gonadotropin-releasing hormone, corticotrophin-releasing hormone, somatostatin, dopamine, calcitonin, adrenaline, noradrenaline, serotonin, human chorionic gonadotropin, insulin, glucagon, erythropoietin, calcitriol, calciferol, atrial natriuretic peptide, gastrin, secretin, cholecystokinin, neuropeptide Y, ghrelin, PYY₃₋₃₆, insulin-like growth factor, angiotensinogen, somatomedin, thrombopoietin, leukotrienes, and prostaglandins (E₁, E₂, F_{1α}, F_{2α}, A₂). In certain preferred embodiments of the invention, hormones that are studied using the methods of the invention include steroid hormones, e.g., glucocorticoids, androgens, estrogens and progestagens. In still more preferred embodiments, such steroid hormones are sexual hormones, preferably, androgens, estrogens and progestagens. In one particularly preferred embodiment, the hormone that is studied using the methods of the invention is DHEA, preferably, in premenarcheal girls. In another preferred embodiment, the hormone is testosterone, preferably, in individuals on a high fat diet and/or having a lifestyle with a lack of physical activity.

The term “bioavailable hormone” includes hormones that can enter into the cells and tissues of a subject without further processing (e.g., enzymatic cleavage), and are not bound to carrier proteins. In preferred embodiments of the methods of the invention, the dynamic hormone index is determined based on the levels of bioavailable hormones in the sample from the subject. In other embodiments, the dynamic hormone index is determined based on the levels of metabolites, degradation products or other proxy indicators of the hormone.

The term “hormone dynamics” includes fluctuations in the levels of one or more hormones in a subject over time. Hormone dynamics may also include comparison of the levels (e.g., ratio, sum, difference) of two or more hormones at the same time.

The term “biological sample” includes samples taken from the subject which may normally or abnormally contain hormones. In certain embodiments of the invention, the biological sample is a body fluid including, but not limited to saliva, blood, urine, mucous, serum, plasma, amniotic fluid, breast ductal fluid, breast milk, tissue aspirates (e.g., needle biopsy), semen, seminal fluid, prostate fluid, lymph, or cerebrospinal fluid. In other embodiments, the biological sample is a tissue sample, for example, skin tissue, bone marrow or tissue biopsy. In still other embodiments, the biological sample includes nail clippings, cheek scrapings, hair or feces. In preferred embodiments, the biological sample contains bioavailable hormones. In some preferred embodiments, the biological sample is selected such that the level of at least one hormone may be measured. In other preferred embodiments, the biological sample is selected such that levels of two or more hormones may be measured from the biological sample.

In particular embodiments, the biological sample is saliva. Saliva collection reduces the invasiveness of sampling relative to venipuncture while providing access to the circulating concentration of steroid hormone (e.g, sex steroid hormone) that is not protein-bound and is therefore able to enter cells and tissues. Saliva provides a readily accessible means of precisely measuring bioavailable hormone (Gann P H et al. J Natl Cancer Inst 1996;88:1118-26) within individuals, repeatedly over time (Berg S J, Wynne-Edwards K E. Mayo Clin Proc 2001;76:582-592; Chiu S et al. Clin Biochem 2003;36:211-4; Lu Y, et al. Fertil Steril 1999;71:863-8; Shirtcliff E A et al. Horm Behav 2000;38:137-147).

The levels of the hormones in the biological sample may be measured by standard techniques that are well known in the art including, but not limited to, immunoassay, gas chromatography, liquid chromatography, mass spectrometry, fluorimetry, and in vitro or in-vivo bio-assays (e.g., measurement of cell, tissue or animal response to a hormone). In particular embodiments the level of the hormone(s) is measured using a commercially available saliva assay kit (e.g., Salimetrics, LLC, State College, Pa.), blood assay kit (Diagnostic Systems Laboratory (DSL) or Diagnostic Products Corp. (DPC)) or urine assay kit (e.g., ZRT Laboratory, Beaverton, Oreg.).

The term “subject” includes organisms that are capable of suffering from or that are potentially at risk of suffering from a hormone-associated condition. Examples of subjects include animals, such as mammals (e.g., a dog, cat, rabbit, ferret, bear, goat, horse, cow, sheep, rat, mouse, non-human primate, transgenic animal or a human). Preferably, the subject is a human. The subject may be male or female. The subject may be prenatal, postnatal, a child (e.g., 0-9 years), an adolescent (e.g., undergoing puberty, 9-18 years, 11-15 years), or an adult (e.g., 18-80 years, 18-40 years, 40-50 years, or 50-80 years, or more than 80 years). In embodiments wherein the subject is a female, the subject may be premenarcheal, postmenarcheal, perimenopausal, or postmenopausal.

The term “population” includes a group of individuals who may share certain attributes or risk factors with the subject. Preferably, the population is made up of individuals who are approximately the same age as the subject, e.g., within 3 months, 6 months, 1 year, 2 years, 3 years, 4 years, 5 years, 6 years, 7 years, 8 years, 9 years, 10 years, 20 years, etc, wherein each number can further represent the upper or lower limit of an age range of individuals in the population relative to the subject. In certain embodiments, the variation of the age of the individuals in the population does not exceed 5%, 10%, 20%, or 30% of the subject's age. In certain embodiments, the population is made up of individuals who share the subject's sex, socioeconomic level, race, ethnicity, body mass index, menarcheal status, physical trait (e.g., height, physical maturity level, etc.) or lifestyle variable (e.g., smoking, drinking, drug use, diet, geographical location, workplace exposure, etc.).

The term “dynamic hormone index” (DHI) is an indicator of the amount of fluctuation of a particular hormone or of the ratio of two or more hormones in a subject or in an individual within a population over a period of time. In certain embodiments, the DHI may be determined by measuring the level of at least one hormone in two or more biological samples obtained from the subject or individual within a population at two or more time points. In other embodiments, the dynamic hormone index is determined by measuring the level of two or more hormones in two or more biological samples obtained from the subject at two or more time points. When two or more hormones are measured, an alternate dynamic hormone index may be measured using the ratio (or sum, or difference) of the two or more hormone levels.

The time points may be selected such that they correspond to the normal temporal pattern for the hormone. These temporal patterns are well-known to those skilled in the art and/or may be readily determined using samples from a normal population. For example, cortisol is known to be at its highest level approximately 45 minutes after awakening, and melatonin is elevated during the night time hours. In certain embodiments of the invention, the time points may occur at intervals of 10 minutes, 30 minutes, hourly, daily, semi-weekly, weekly, bi-weekly, tri-weekly, monthly, bimonthly, quarterly, semi-annually, yearly. The biological samples may be obtained, for example, over a period of about one day, one week, two weeks, three weeks, four weeks, six weeks, and eight weeks, three months, 6 months, one year, two years, or five years.

The dynamic hormone index may be calculated for a particular subject or individual in a population by using any appropriate statistical analysis or mathematical algorithm. In one exemplary embodiment, the dynamic hormone index is calculated using the co-efficient of variation (CV):
CV=(standard deviation/mean)*100

The CV may be corrected for sample size of less than 30 by the following formula:
Corrected CV=CV*(1+(1/(4N)))
Wherein N is the number of the individuals in the population.

The phrase “statistical distribution of a dynamic hormone index” refers to the range of values for a dynamic hormone index among the population. The statistical distribution may be parametric or non-parametric. Preferably, the statistical distribution is a normal distribution (i.e., continuous or interval-scale data), but may also be a binomial distribution or Poisson distribution. Transformation of hormone values by methods well known to those of skill in the art, including, in a preferred embodiment, logarithmic transformation is sometimes advantageous for statistical comparisons. The statistical distribution for any dynamic hormone index may be calculated from a population using standard statistical analyses well-known to those of skill in the art such as the tests described herein and, including, but not limited to, Student's t, Fisher's F, and Pearson's r.

The terms “mean dynamic hormone index” and “median dynamic hormone index” are used herein in a manner consistent with the well-understood definitions in the art of statistics. For example, the mean is the average value of the dynamic hormone indices of a population of individuals, whereas the median refers to the value halfway through the dynamic hormone indices of a population, below and above which there lie an equal number of data values. Preferably, the population comprises 20 or more, 30 or more, 40 or more, 50 or more, 75 or more, 100 or more, 200 or more, 300 or more, 500 or more or 1000 or more individuals. Furthermore, in certain circumstances, for example, related to an established public health issue (e.g., Harvard Nurses Study II, Wu et al., J Natl Cancer Inst 1999;91:529-34; Cho et al., J. Natl. Cancer Inst., 95:1079-1085, 2003; Baer et al., Cancer Epidemiol. Biomarkers Prevent., 12: 159-1167, 2003), the population may be more than 10,000 individuals, and may be 90,000 or more individuals. The methodology used for determining the individual dynamic hormone indices which make up the mean or median dynamic hormone index may be the same as that used for the subject or different. In certain embodiments of the invention, the mean or median dynamic hormone index may be obtained from a data bank corresponding to currently accepted normal levels of the hormones under analysis and/or normal dynamic hormone indices of the hormone(s) under analysis.

The comparison or “statistical relationship” of the dynamic hormone index of an individual can be a straightforward comparison, such as a ratio, or it can involve weighting of one or more of the measures relative to, for example, their importance to the particular situation under consideration. The comparison can also involve subjecting the dynamic hormone index and/or the mean dynamic hormone index to any appropriate statistical analysis. It is important to note that it is possible that one or more of the measures obtained will render an inconclusive result; accordingly, data obtained from a battery of measures can provide for a more conclusive result. It is for this reason that an interpretation of the data based on an appropriate weighting scheme and/or statistical analysis is desirable.

A “statistically meaningful relationship,” as used herein, refers to an association between two or more of the described parameters and/or hormone-associated condition(s) of the invention that deviates from what might be considered independence or randomness. The parameters and/or hormone-associated conditions of the invention include dynamic hormone indices, risk factors, hormone-associated conditions and/or symptoms or adverse side effects of a hormone-associated condition. The relationship is characterized informally by some kind of correlation or non-random co-occurrence or co-variation. More formally, a statistically meaningful relationship may be measured or described in terms of any of a number of mathematical concepts derived from statistics, probability theory, information theory, and/or data mining. For example, in some embodiments, statistically meaningful refers to statistically significant, in terms of high numbers of co-occurrences in a theoretical series of randomized trials, as in 19 times out of 20, 99 times out of 100, 999 times out of 1000, etc., and may also be phrased in the terms commonly used in hypothesis tests such as alpha being less than 0.05, less than 0.01, less than 0.001, less than 0.0005, etc. In some embodiments, significantly meaningful refers to the rejection of a null hypothesis of no significant relationship, the rejection being phrased in terms of confidence intervals. In some embodiments, significantly meaningful refers to specific tertile(s), quartiles, quintiles relative to the reference population, and might be presented in the form of a relative risk ratio. In some embodiments, significantly meaningful may also refer to a value that falls outside the 99% or the 95% or the 90% etc confidence interval for a reference measure. In some embodiments, it may refer to the measure in a population that is able to meaningfully explain a portion of the variance in the population, as, for example, a father's adult height might predict his son's height, or the date on the calendar might predict the noon-time temperature, or knowledge of a coin toss weighting or bias might predict whether heads or tails will be seen next in a series of coin toss. In such cases, the statistics might be reported as, for example, coefficients of correlation, or degrees of goodness-of-fit, or equations derived from multiple regression, or the first one or more principal components derived in a principal components analysis. Or they may be reported as values known to those in the art that are associated with particular forms of hypothesis testing, such as the Student s t test or Chi-squared test. In some embodiments, the statistically meaningful relationship or relationships might be non-linear, as in, for example, the Matthews or four-point correlation measure, or the degree of mutual information as used in information theory approaches. In some nonlinear measurements of statistical dependency, the reported statistics takes the form of a step function, where the relationship moves from a correlation of zero to a correlation of 1. Statistical dependencies or other meaningful relationships can also be reported in terms of the parameters of probability distributions, such as a Poisson distribution.

In the methods of the invention, the dynamic hormone index for the subject is compared to the dynamic hormone index of a population by statistical placement of the dynamic hormone index of the subject along the continuum of dynamic hormone index for the population. In certain embodiments, the dynamic hormone index of a hormone is determined by measuring the level of the hormone in two or more samples from the subject at two or more time points. In other embodiments, the dynamic hormone index is determined by measuring the level of two or more hormones in two or more biological samples obtained from the subject at two or more time points. When the levels of two or more different hormones are compared, the ratio of the levels may be compared directly to the statistical distribution of the same hormone(s) of a population, or two or more samples may be taken at different points in time and the fluctuations of the ratios of the two or more hormones may be studied over time using a co-efficient of variability such as described above. The co-efficient of variability for the ratios of the two or more hormones may then be compared to a statistical distribution of the dynamic hormone index of a population which has had similar measurements and ratios calculated.

The term “aberrant dynamic hormone index” includes hormone indices whose placement on the statistical distribution of the dynamic hormone index of the population indicates a statistically meaningful difference from the mean or median dynamic hormone index of the population, or a within-individual change in DHI (e.g., in response to treatment), and is indicative that the subject is at risk of developing a hormone associated condition. For example, an aberrant dynamic hormone index may vary by more than 5%, 10%, 25%, 30%, 35%, 40%, 45%, 50%, 55%, 60%, 65%, 70%, 60%, 90% or 100%, 200%, 300% or more from the mean or median dynamic hormone index of the population, wherein each number can further represent the upper or lower limit of a range of percentages by more than which the dynamic hormone index may vary (e.g., 5-100%, 25-100%, 50-200%, etc). In another embodiment, an aberrant dynamic hormone index varies one or more, two or more, three or more or four or more standard deviations away from the mean dynamic hormone index. In certain preferred embodiments, the level of variance between the subject's dynamic hormone index and the mean or median dynamic hormone index of the population is indicative of the level of risk of developing a hormone-associated condition in the subject.

The term “hormone-associated condition” includes conditions which are stimulated by, inhibited by, or otherwise associated with hormones. Examples of hormone-associated conditions include cancers, such as breast cancer, ovarian cancer, uterine cancer, cervical cancer, prostate cancer, testicular cancer, thyroid cancer, parathyroid cancer, adrenal gland cancer, colon cancer and pancreatic cancer. Other hormone-associated conditions include diabetes (e.g., juvenile diabetes, type I diabetes, type II diabetes), metabolic syndrome X, obesity, stress, symptoms of aging, hyperthyroidism, hypothyroidism, cardiovascular disease (e.g., high blood pressure), high cholesterol and inflammation.

The term “metabolic syndrome X or MSX” includes a combination of three or more of the following symptoms: abdominal obesity, elevated triglyceride levels, decreased high-density lipoprotein (HDL) cholesterol levels, high blood pressure, and impaired fasting blood glucose (a measure for decreased insulin sensitivity and increased risk of developing diabetes). Metabolic syndrome X is associated with several metabolic abnormalities which may be related to insulin resistance and/or impaired glucose tolerance (Hansen, BC (1999) Ann NY Acad Sci 892:1). Metabolic syndrome X significantly increases the risk of coronary heart disease (CHD) and atherogenesis, and has been identified as an independent target for coronary heart disease risk reduction, separate from low-density lipoprotein (LDL) cholesterol elevation. Metabolic syndrome X is prevalent, with estimates as high as one in four US adults (Ford, E S, et. al. (2002) JAMA 287:356-359).

In another aspect, the invention provides a method of identifying a modifiable risk factor that has a statistically meaningful relationship with a hormone-associated condition. The method includes determining the dynamic hormone index of at least one hormone for individual subjects of a population, and determining the level of exposure for individual subjects of the population to a suspected modifiable risk factor. In this method, a statistically meaningful relationship between an aberrant dynamic hormone index of subset of the subjects in the population and the level of exposure of the subset to the risk factor is indicative that exposure to the risk factor increases the likelihood of developing a hormone-associated condition.

The statistically meaningful relationship between a risk factor and a hormone-associated condition or dynamic hormone index may be either parametric or nonparametric. The statistically meaningful relationship between a risk factor and a hormone-associated condition may be linear or non-linear. In certain preferred embodiments, the level of exposure to the risk factor correlates with dynamic hormone index and/or hormone-associated condition, and may have a positive or negative association, e.g., correlation.

The term “modifiable risk factor” includes risk factors for which exposure may be altered by a change in the subject's behavior, habits or environment. For example, modifiable risk factors include, but are not limited to smoking, alcohol consumption, drug use, diet, physical activity, body mass index, high percentage of animal fat intake or consumption, geographic location, and workplace environment.

For example, a number of recent studies have moved physical inactivity from a suspected (Bernstein L et al. J Natl Cancer Inst 1994;86: 1403-8) to a known risk factor for breast cancer (Friedenreich C M et al. Am J Epidemiol 2001;154:336-47; Friedenreich C M, et al. Epidemiology 2001;12:604-12; Friedenreich C M, et al. Med Sci Sports Exerc 2001;33:153845; Yang D et al. Cancer 2003;97:2565-75). As physical activity is modifiable, and interventions early in life are most likely to have lasting effects on lifetime activity, associations between activity variables and hormone exposures are important components of primary prevention strategies (Friedenreich C et al. Chronic Dis Can 2001;22:41-9). In one recent study, recall of physical activity at age 16 was inversely associated with premenopausal breast cancer risk (Dorn J et al. Med Sci Sports Exerc 2003;35:278-85). Physical inactivity is also an emerging epidemic in the western world (Tremblay M S et al. Int J Obes Relat Metab Disord 2002;26:538-543), and is linked to other hormone-associated conditions such as metabolic syndrome X and obesity (Katzmarzyk P T et al. J Clin Epidemiol 2001;54: 190-5), coronary heart disease (Katzmarzyk P T et al. Prev Med 1999;29:555-62), and adolescent musculoskeletal condition (Katzmarzyk P T et al. Med Sci Sports Exerc 1998;30:709-14).

In another aspect, the invention provides a method of determining whether there is a statistically meaningful relationship between a known risk factor and an aberrant dynamic hormone index. The method includes determining the dynamic hormone index of at least one hormone for individual subjects of a population, and determining the level of exposure for the individual subjects of the population to the risk factor. In this method, a statistically meaningful relationship between an aberrant dynamic hormone index of a subset of the subjects in the population and the level of exposure of the subset to the risk factor is indicative that there is a relationship between the level of exposure to the risk factor and the dynamic hormone index. In preferred embodiments, the aberrant dynamic hormone index correlates, either positively or negatively, with an increase in exposure to the risk factor.

In another aspect, the invention provides a method of reducing the risk of developing a hormone-associated condition in a subject identified as having an aberrant dynamic hormone index, by altering the level of exposure of the subject to at least one modifiable risk factor associated with the hormone-associated disorder. In one preferred embodiment, the exposure to the modifiable risk factor of the subject is reduced.

In yet another aspect, the invention provides a method of monitoring a subject's risk of developing a hormone-associated condition wherein the subject has an aberrant dynamic hormone index. The method includes altering the exposure of the subject to at least one modifiable risk factor, and determining the dynamic hormone index of the subject after the exposure to the risk factor has been altered. In this method, a shift of the dynamic hormone index of the subject in a direction associated with lower health risk is indicative that the risk of the subject developing the hormone-associated condition is reduced. In certain embodiments, such a shift is toward the mean or median dynamic hormone index of the population.

In yet another aspect, the invention provides a method of identifying a course of treatment for reducing the risk of developing a hormone-associated condition in a subject. The method includes monitoring the dynamic hormone index of a subject having an aberrant hormone index during the course of treatment. In this method, a shift of the dynamic hormone index of the subject in a direction associated with lower health risk during the course of treatment is indicative that the treatment reduces the subject's risk of developing the hormone-associated condition. In certain embodiments, such a shift is toward the mean or median dynamic hormone index of the population. In some embodiments, the treatment alters the exposure of the subject to a risk factor and/or comprises hormone therapy. In some embodiments, the treatment includes administering an agent that alters the level of hormone in the subject. For example, in certain embodiments the agent may be the same hormone or another hormone (e.g., phytoestrogens).

The invention also provides a method of determining whether a subject has an increased risk of developing one or more symptoms or adverse side effects of a hormone-associated condition, by determining the relative level of at least one hormone in two or more biological samples from the subject taken at two or more time points, and determining a dynamic hormone index for the subject. In this method, a dynamic hormone index for the subject that is aberrant when compared to the statistical distribution of the dynamic hormone index of the population is indicative that the subject has an increased risk of developing one or more symptoms or adverse side effects of a hormone-associated condition (e.g., palpitations, depression, mood swings, vaginal dryness, osteoporosis, blindness, cardiovascular disease, morbid obesity). In one preferred embodiment, the dynamic hormone index of the subject is compared to the mean or median dynamic hormone index of the population, and aberrant is at least 25%, and preferably at least 50% greater than the mean or median dynamic hormone index for the population.

In yet another aspect, the invention provides a method of determining the level of risk for developing a hormone-associated condition in a subject. In certain embodiments, the method includes determining the level of at least one hormone in two or more biological samples taken from the subject at two or more time points, and determining a dynamic hormone index for the subject. In other embodiments, the dynamic hormone index is determined by measuring the ratio of two or more hormones in two or more samples obtained from the subject at two or more time points. In this method, the level of variance between the subject's dynamic hormone index and the mean or median is indicative of the level of risk for developing the hormone-associated condition in the subject.

In yet another aspect, the invention provides a kit for determining whether a subject has an increased risk of developing a hormone-associated condition. The kit includes means for determining the relative level of at least one hormone in at least one sample from the subject, and instructions for identifying whether the individual is at risk of developing the hormone-associated condition associated with or indicated by the measured hormone or its dynamic index. In other embodiments, the kit measures the amounts of two hormones and compares the ratio of the hormones to a standard. The kit may further comprise means for determining the relative level of at least one hormone in two or more samples from the subject taken at two or more time points. In preferred embodiments, the kit includes means for determining dynamic hormone index of at least one predictive hormone for which relative level(s) in sample(s) is determined. The means for determining the dynamic hormone index may include means for reporting the dynamic hormone index. In some embodiments, the reporting means may include an indicator value or range for determining whether the measured hormone levels or above or below the mean or median dynamic hormone index of the population (e.g., a colorimetric indicator). In some embodiments, the reporting means may include an indicator value or range that measures a statistically meaningful difference between the measured dynamic hormone difference and the mean or median dynamic hormone index of the population. In some embodiments, the kit may contain instructions which comprise means for calculating the mean dynamic hormone index (e.g., according to a mathematical algorithm). In some embodiments, the kit may contain a reference to an internet site or other databank that provides means for determining whether relative level of the hormone is indicative of an aberrant dynamic hormone index. For example, in certain embodiments the databank may include the statistical distribution of one or more dynamic hormone indices of the population, and may also show the mean and/or median dynamic hormone indices for the relevant population.

### EXEMPLIFICATION OF THE INVENTION

This invention is further illustrated by the following examples which should not be construed as limiting.

### Example 1

### Association of Risk Factors with Dynamic Hormone Index

### Lifestyle and Hormone Dynamics in Adolescent Girls

Individual differences in the amplitude and pattern of sex steroid hormone variability will alter hormone receptor binding and the activity of second messenger pathways within cells. Within limits, each doubling of the hormone concentration bathing a cell will double the number of available hormone receptors that are activated by hormone binding. Thus, two individuals with the same average hormone concentration over time might differ profoundly in the impact of that average hormone concentration on their cells. Cells responding to regular (or irregular) halving or doubling in the hormone concentration will not be in the same physiological state as cells in equilibrium with a stable hormone signal. Thus, the magnitude of intra-individual steroid hormone variability through time might have more impact on cellular biochemistry than the average concentration.

We hypothesized that inter-individual differences in the extent of intra-individual sex steroid hormone variability would be associated with risk for hormone-sensitive diseases including breast cancer. No published studies have yet considered the extent of day-to-day, intra-individual, variability in sex steroid hormone concentration, i.e., a dynamic hormone index (DHI), as a possible biomarker for breast cancer. However, individual heart rate variability has recently proven clinically relevant in heart disease, chronic fatigue syndrome, and physical fitness (Javorka et al., Clin. Physiol. Funct. Imaging, 23: 1-8, 2003; Karcz et al., Int. J. Cardiol., 87: 75-81, 2003; Singh et al., Am. J. Cardiol., 90: 1290-1293, 2002; Yamamoto et al., Exp. Biol. Med., 228: 167-174, 2003).

The study described below was directed at understanding the interaction of lifestyle and environmental risk factors for breast cancer with individual hormone dynamics. Lifestyle factors linked to the risk of breast cancer include physical activity (Friedenreich et al, Cancer, 83: 600-610, 1998; Thune et al., Med. Sci. Sports Exercise, 33: S530-S550, 2001), body composition (Calle, et al., N. Engl. J. Med., 348: 1625-1638, 2003; Huang et al., J. Am. Med. Assoc., 278: 1407-1411, 1997; Petrelli et al., Cancer Causes Control, 13: 325-332, 2002), hormone replacement therapy (Chlebowski et al., J. Am. Med. Assoc., 289: 3243-3253, 2003), dietary animal fat intake (Cho et al., J. Natl. Cancer Inst., 95: 1079-1085, 2003), and alcohol consumption (Hunter et al., Epidemiol. Rev., 15: 110-132, 1993). These anthropometric and lifestyle variables might affect breast cancer risk by altering individual exposure to sex steroids including estradiol, progesterone, testosterone, and DHEA (dehydroepiandrosterone) (Chlebowski et al., J. Am. Med. Assoc., 289: 3243-3253, 2003; Dorgan et al., J. Natl. Cancer Inst., 93, 710-715. 2001; Gustafsson et al, J. Steroid Biochem. Molec. Biol., 74: 245-248, 2000; Lillie et al., Breast Cancer Res., 5: 164-173, 2003; Berrino et al., J. Natl. Cancer Inst., 88: 291-296, 1996; Cauley et al., Ann. Internal Med., 130: 270-277, 1999; Key et al. J. Natl. Cancer Inst., 95, 1218-1226.2003; McTiernan et al., J. Clin. Oncol., 21: 1961-1966, 2003; Morris et al., Surgery, 130: 947-953, 2001; Reichman et al., J. Natl. Cancer Inst., 85: 722-727, 1993). The current study is the first to quantify the dynamic index for sex steroid hormones, and the first to determine the distribution of that dynamic index in a population.

The subjects of the research were adolescent girls. During adolescence, hormones are active in breast development (Brisken et al., J. Mammary Gland Biol. Neoplasia, 7: 39-48, 2002), adult menstrual cycles become established (Apter et al., J. Clin. Endocrinol. Metab., 57: 82-86, 1983), breast tissue contains steroid hormone receptors (Pike et al., Epidemiol. Reviews, 15: 17-35, 1993; Neville et al., J. Mammary Gland Biol. Neoplasia, 7: 49-66, 2002), and breast tissue has a high intrinsic cell division rate (Russo et al., Breast Cancer Res. Treat., 2: 5-73, 1982). Adolescent lifestyle variables are risk factors for breast cancer (Frazier et al., Breast Cancer Res., 5: R59-R64, 2003; Okasha et al., Breast Cancer Res. Treat., 78: 223-276, 2003; Romundstad et al., Int. J. Cancer, 105: 400403, 2003) and might provide important insights into the mechanisms linking lifestyle characteristics with biological risk (Okasha et al., Breast Cancer Res. Treat., 78: 223-276, 2003; Lawlor et al., British Journal of Cancer, 89: 81-87, 2003). Some adolescent risks for later breast cancer are not modifiable, such as the age at menarche (Bernstein et al., Neoplasia, 7: 3-15, 2002; Hamilton et al., N. Engl. J. Med., 348, 2313-2322, 2003) and the age when adult height is attained (Romundstad et al., Int. J. Cancer, 105: 400-403, 2003; Li et al., Epidemiology, 8: 559-565, 1997; Hilakivi-Clarke et al., Br. J. Cancer, 85: 1680-1684, 2001). Other adolescent risks for later breast cancer, including low levels of physical activity and dietary consumption of animal fat (Frazier et al., Breast Cancer Res., 5: R59-R64, 2003; Katzmarzyk et al., Preventive Med., 29: 555-562, 1999; Dorn et al., Med. Sci. Sports Exercise, 35: 278-285, 2003; Baer et al., Cancer Epidemiol. Biomarkers Prevent., 12: 1159-1167, 2003), are modifiable and thus have the most potential for primary prevention of disease through behavioral modification (Okasha et al., Breast Cancer Res. Treat., 78: 223-276, 2003; Friedenreich et al., Chronic Disease Can., 22: 41-49, 2001).

Based on these established links between adolescence and breast cancer, the current study assessed the lifestyle of each participant in terms of anthropometry, diet, and physical activity. Anthropometric variables were age-adjusted height, body mass index, and percent body fat. Early attainment of adult height during adolescence is associated with increased breast cancer risk (Okasha et al., Breast Cancer Res. Treat., 78: 223-276, 2003; Romundstad et al., Int. J. Cancer, 105: 400-403, 2003; Lawlor et al., British Journal of Cancer, 89: 81-87, 2003; Li et al., Epidemiology, 8: 559-565, 1997; Hilakivi-Clarke et al., Br. J. Cancer, 85: 1680-1684, 2001). Body mass index and percent body fat change predictably with puberty and were included as measures of sexual maturation (van Hooff et al., J. Clin. Endocrinol. Metab., 85, 1394-1400. 2000; Lindsay et al., J. Clin. Endocrinol. Metab., 86, 4061-4067. 2001). To minimize both the number of variables being considered and the potential for confounding across dietary variables, dietary animal fat was chosen as the sole dietary risk factor. This choice was based on recent results from the prospective Nurses Health Study II. In 2003, this study quantified the relative risk of premenopausal breast cancer relative to dietary fat and concluded that animal fat intake was the primary association (95% CI=1.02-1.73) (Cho, E et al., J. Natl. Cancer Inst., 95: 1079-1085, 2003). That result was recently extended to a positive association between adolescent animal fat intake and benign breast disease (Baer et al., Cancer Epidemiol. Biomarkers Prevent., 12: 1159-1167, 2003). The choice was also based upon a recent study that found adolescent sex steroid hormone changes after a behavioral intervention that reduced fat intake (Dorgan et al., J. Natl. Cancer Inst., 95, 132-141. 2003). Two measures of physical activity, strenuous leisure activity and leisure inactivity, were used. Adolescent physical activity has an inverse association with breast cancer (Dorn et al., Med. Sci. Sports Exercise, 35: 278-285, 2003). These associations are well established in adult populations (Bernstein et al., J. Natl. Cancer Inst., 86: 1403-1408, 1994; Friedenreich et al., Am. J. Epidemiol., 154: 336-347, 2001; Friedenreich et al., Epidemiology, 12: 604-612, 2001; Friedenreich et al., Med. Sci. Sports Exercise, 33: 1538-1545, 2001; Yang et al., Cancer, 97: 2565-2575, 2003).

In the current study, salivary hormone determination methods minimized the invasiveness of sample collection and thereby provided a readily accessible means of precisely measuring the bioavailable sex steroid hormone concentration (hormone that is not bound to a carrier protein and is therefore biologically available to cells) within individuals, over time (Berg et al., Mayo Clinic Proc., 76: 582-592, 2001; Gann et al., J. Natl. Cancer Inst., 88: 1118-1126, 1996; Chiu et al., Clin. Biochem., 36: 211-214, 2003; Lu et al., Fertil. Steril., 71: 863-868, 1999; Shirtcliff et al., Horm. Behav., 38: 137-147, 2000). Samples were analyzed for estradiol, progesterone, testosterone, and DHEA concentration. Each hormone is actively modulated during adolescence in girls (Apter et al., J. Clin. Endocrinol. Metab., 57: 82-86, 1983; van Hooff et al., J. Clin. Endocrinol. Metab., 85, 1394-1400. 2000; Dorgan et al., J. Natl. Cancer Inst., 95, 132-141. 2003; Ankarberg et al., J. Clin. Endocrinol. Metab., 84, 975-984. 1999; Norjavaara et al., J. Clin. Endocrinol. Metab., 81, 4095-4102. 1996; Groschl et al., Fertil. Steril., 76, 615-617. 2001; Reading et al., Int. J. Sport Nutr. Exercise Metab., 12: 93-104, 2002) and evidence in adult women links each to breast cancer risk. (Dorgan et al., J. Natl. Cancer Inst., 93, 710-715. 2001; Dorgan et al., J. Natl. Cancer Inst., 94: 606-616, 2002; Lamar et al., Cancer Epidemiol. Biomarkers Prevent., 12: 380-383, 2003). During adolescence, estradiol and progesterone establish menstrual rhythms in their concentrations through time. The androgens (testosterone and DHEA) do not establish menstrual rhythms, but are linked to estradiol and progesterone by a shared biosynthetic pathway. Androgens are synthesized from progesterone and estradiol is synthesized from androgens.

There were two specific objectives. The first objective was to quantify day-to-day hormone variability through calculation of a dynamic index and to determine the range of variability among adolescent girls. The second was to explore possible relationships between the dynamic index and adolescent anthropometric and lifestyle variables linked to breast cancer risk in adulthood.

Materials and Methods

Setting, Recruitment, and Participants

Participants were recruited at a private girl's school in Ontario, Canada. Students were invited to participate via information forms sent home, using procedures that were approved by the Research Ethics Board of Queen's University (Biol 003-02). Grades for recruitment were chosen to represent hormone dynamics before the first menstrual period (Grade 5), a split between girls with and without menstrual periods (Grade 8), and girls with regular menstrual cycles (Grade 11). Most girls (93%) reported their ethnic origin as white, or white plus an additional ethnic or cultural background. The majority reported no history of smoking (95%) and none smoked on a daily basis. Alcohol consumption was minimal. Of 151 eligible participants, 94 returned signed consent forms, yielding at least the expected consent rate for communication materials sent home with children (Sallis et al., Med. Sci., Sports Exercise, 28: 840-851, 1996). After screening for completeness of hormone analysis, interview, anthropometric, physical activity, and dietary data, 56 participants were included in these analyses. Of those, 22 were premenarcheal and 34 were postmenarcheal.

Data Collection

Food Frequency Questionnaire (FFQ)

A previously validated youth/adolescent FFQ developed at the Channing Laboratory at Harvard University was used (Frazier et al., Breast Cancer Res., 5: R59-R64, 2003; Rockett et al., Preventive Med., 26: 808-816, 1997; Rockett et al., Am. J. Clin. Nutr., 65: 1116S-1122S, 1997; Rockett et al., J. Am. Dietetic Assoc., 95: 336-340, 1995). Questions were asked about food frequency over the past year, but were phrased in terms of typical intake on a daily or weekly basis, yielding a total dietary intake in kcal/day. Only one dietary variable, proportion of daily caloric intake derived from animal fat (dietary animal fat %) was included as a lifestyle variable in this analysis.

Physical Activity Survey (PAS)

The PAS was also provided and coded by the Channing Laboratory at Harvard University. It consisted of 24 items. Twenty questions were about specific types of physical activity (from walking to school to skateboarding) over the past year and each question offered six choices for the level of participation (range: 0-6+ hours per week). Physical inactivity was based on reports of the total time per weekday or weekend day spent on a range of activities from ‘hanging out with friends’ to playing computer games. Two summary variables, hours of physical activity and hours of physical inactivity per week, were generated. Similar physical activity surveys have been used by others (e.g., (Kowalski et al., Pediatric Exercise Science 1997;9:343-353; Kowalski et al., Pediatric Exercise Science 1997;9;1740186)

Telephone Interview

A telephone interview was conducted by one of two trained interviewers during the school day. A private office with a telephone was provided by the school and participants were released from class at pre-arranged times to receive the interviewer's call. Age, ethnicity, oral contraceptive use, and exposure to cigarette smoke as well as Tanner stage of breast and pubic hair development (based on five illustrations validated for this age group (Taylor et al., Paed. Perinatal Epidemiol., 15: 88-94, 2001) were recorded. If postmenarcheal, participants were asked when menarche occurred; how many days had elapsed between the first day of their last menstrual bleeding and the first day of the previous menstrual bleeding; and the duration (days) of their last menstrual bleeding. Age, gynecological age (months since menarche), breast development and pubic hair were used in the analysis to control for sexual maturation. Each interview lasted about approximately 12 minutes.

Anthropometry

A trained volunteer was responsible for all body measurement. Height (cm) was measured using a wall-chart. Body weight (kg) and body fat composition (% body fat) were measured using the Tanita Model 350 BF impedance body composition analyzer as validated for use in children from ages 6 to 18 years. Body mass index (BMI) was calculated as weight (kg)/height (m²).

Saliva Collection

Saliva collection occurred on each of 28 school days over a six-week interval. The number of saliva samples varied by participant according to days away from school at morning recess (e.g. athletic competitions, examinations, sickness) as well as insufficient volume (Table 1). Samples were collected at the beginning of morning recess to: a) control for circadian changes in hormone concentrations (Ankarberg et al., J. Clin. Endocrinol. Metab., 84, 975-984. 1999; Norjavaara et al., J. Clin. Endocrinol. Metab., 81, 4095-4102. 1996), b) minimize disruption to the school schedule, c) minimize the potential for blood contamination from recent brushing of teeth, and d) reduce food particulate in the mouth. Saliva was collected by passive drool into a ‘Salivette’ (Sarstedt, QC) from which the absorbent cotton and carrying insert had been removed. Although cotton does not interfere with all salivary assays for steroid hormones there is evidence to suggest that estradiol and DHEA assays can be adversely affected (Shirtcliff et al., Psychoneuroendocrinology, 26: 165-173, 2001). A volunteer coordinator from the school oversaw saliva collection and promptly froze samples. Three ml of saliva each day were requested (although less would have been sufficient) and samples were stored frozen (−20° C.) until hormone analysis.

TABLE 1

| | | Estrogen | | Progesterone | | Testosterone | | DHEA | |
|---|---|---|---|---|---|---|---|---|---|
| | N | Median | Range | Median | Range | Median | Range | Median | Range |
| Premenarcheal | 22 | 8 | 3-24 | 16 | 8-27 | 15 | 8-27 | 16 | 8-27 |
| Postmenarcheal | 34 | 19 | 8-26 | 23 | 16-27 | 22 | 16-26 | 23 | 16-27 |

Hormone Determinations

To minimize freeze-thaw effects, all four hormones from any sample were analyzed in the same day. To minimize the contribution of inter-assay variability to intra-individual hormone comparisons, all samples for one individual were placed on the same plate. Samples were thawed under gentle shaking in a water bath at 20° C. and then centrifuged (1500×g, 15 min, 18° C.) to precipitate mucins and detritus from the samples. The resulting supernatant was used in all hormone assays. Concentrations of estradiol (17β-estradiol), progesterone, testosterone, and DHEA were determined using enzyme-linked immunoassay (EIA) kits designed for use with saliva (Salimetrics LLC, State College, Pa.). Triplicate determinations of high and low controls were distributed across each plate. If more than two of the six determinations were not in the acceptable range, the plate was rejected. When determinations fell below the lowest standard, concentrations were rounded up to the lowest standard (Table 2). In practice, these adjustments were rare, affecting only 151/1058 estradiol (14%), 188 of 1516 progesterone (12%), 7 of 1413 testosterone (0.5%), and 57 of 1512 DHEA (4%) samples. For progesterone, testosterone, and DHEA, at least 20% of saliva samples were assayed in duplicate, with the rest as singletons. Duplicate determinations were limited only by the need to quantify all samples from one individual on the same 96-well plate. As the estradiol assay was working at low concentrations and was not limited by well availability, all samples with sufficient volume were assayed in duplicate. In practice, 80% of samples had sufficient volume to be assayed as duplicates. Except within 10% of the lower limit of assay sensitivity, any dynamic index greater than 15% was repeated as a duplicate on a subsequent plate. Repetitions were used to reject one of the two original duplicates. This approach minimized measurement error in estradiol quantification. Performance characteristics of the assays and the internal quality controls are summarized in Table 2.

TABLE 2
Details of salivary hormone assay controls and resulting
measures of sensitivity, accuracy, and precision.

| | Sensitivity | | | | | | |
|---|---|---|---|---|---|---|---|
| | range | Control | Expected Control | Assays | Measured Controls | Intra-assay | Inter-assay |
| Hormone | (pg/ml) | Batch* | Range (pg/ml)** | (#) | (pg/ml ± SE) | DHI (%) | DHI (%) |
| Estradiol | 1-64 | A | H = 30.56-5.84 | 12 | H = 34.0 ± 1.5 | H = 12.9 | H = 23.5 |
| | | | L = 3.72-5.58 | | L = 5.5 ± 0.5 | L = 17.8 | L = 47.7 |
| | | B | H = 30.56-45.84 | 16 | H = 43.5 ± 0.8 | H = 8.4 | H = 13.5 |
| | | | L = 2.7-5.9 | | L = 5.2 ± 0.2 | L = 13.6 | L = 26.3 |
| Progesterone | 10-1000 | A | H = 3^{rd} trimester† | 17 | H = 639.1 ± 17.5 | H = 7.7 | H = 11.3 |
| | | | L = post-partum | | L = 31.7 ± 1.5 | L = 17.5 | L = 22 |
| | | B | H = 500†† | 15 | H = 467.5 ± 5.0 | H = 3.7 | H = 6.8 |
| | | | L = 35 | | L = 48.5 ± 1.8 | L = 14.5 | L = 22.2 |
| Testosterone | 1.5-360 | A | H = 162-243 | 17 | H = 211.3 ± 4.3 | H = 5.1 | H = 11.3 |
| | | | L = 11.6-17.4 | | L = 14.8 ± 0.6 | L = 15.5 | L = 22.7 |
| | | B | H = 155.3-233.3 | 15 | H = 188.1 ± 3.7 | H = 7.0 | H = 11.9 |
| | | | L = 10.2-15.2 | | L = 13.1 ± 0.4 | L = 9.8 | L = 17.7 |
| DHEA | 10-1000 | A | H = 522-782 | 18 | 546 ± 10 | H = 5.5 | H = 10.6 |
| | | | L = 38.2-57.4 | | 47.7 ± 1.2 | L = 10.6 | L = 14.7 |
| | | B | H = 424-636 | 15 | 473.1 ± 7.3 | H = 5.5 | H = 10.2 |
| | | | L = 40-60 | | 36.5 ± 1.4 | L = 8.5 | L = 25.2 |
| *A and B denote two quality control batches. One or the other batch was quantified in triplicate during each assay run. | | | | | | | |
| **H and L denote controls at the high end of the sample range (H) and the low end of the sample range (L). | | | | | | | |
| †Progesterone controls were not available with the commercial EIA kit. In batch 1, controls were previously frozen aliquots of supernatant from a pooled sample from a single individual during weeks 36-39 of a full-term pregnancy (H) and the same individual during weeks 2-4 postpartum (L). Expected concentrations were not known. Measured concentration is shown in Column 4. | | | | | | | |
| # After the first batch of control aliquots was exhausted, alternative control pools were created in a saliva matrix. A saliva pool (male and female, only oral contraceptive use was excluded) was collected and dextran-coated charcoal was used to adsorb steroids during vigorous mixing at 4° C. The charcoal-stripped supernatant was then spiked with progesterone (Injectable, pharmaceutical grade, in a saline vehicle) and saved as aliquots to create the H and L controls. | | | | | | | |

Statistical Analysis

A mean hormone concentration was calculated across all samples for each participant. As a result of the multiple determinations contributing to the mean hormone concentration for each participant (Table 1), measurement error in the mean estimates was expected to be small. Aggregation by participant also eliminated extreme values, yielding approximate normal distributions across participants and no requirement to transform hormone results prior to statistical analyses.

A dynamic hormone index, specifically, a coefficient of variation, CV (CV=[standard deviation/mean]*100), was calculated for each participant. The dynamic index was then corrected for underestimated standard deviation because N <30 [Corrected dynamic index=CV*(1+(1/(4N)))] (Sokal, R. R. and Rohlf, F. J. Biometry. United States of America: W.H. Freeman and Company, 1981). This method of calculating a dynamic index was chosen to reduce the impact of outlying hormone values relative to alternative calculations, such as absolute range, that are extremely sensitive to outlying values. Reduced sensitivity to outliers was the direct result of integrating all sample concentrations into the standard deviation used to calculate the dynamic index for each participant. Standard deviation was then adjusted by the mean concentration to yield coefficient of variation to ensure that intrinsic differences in assay precision at different points on the standard curve (different absolute concentrations) would not be reflected in different absolute magnitudes for the standard deviation. Any such association would have resulted in spurious associations between the hormone concentration and the dynamic index in response to measurement error rather than individual differences. Measurement error was further reduced because all samples were quantified on the same EIA plate, so that only intra-assay measurement error contributed to the dynamic index calculation.

All variables were examined for outliers, and values beyond +/−3 standard deviations were eliminated. Five outliers (one for estradiol, one for progesterone, one for DHEA, and two for physical inactivity) were excluded.

Descriptive statistics were computed, and differences in mean between premenarcheal and postmenarcheal participants were assessed using a t-test. Mean estradiol concentration did not increase from the premenarcheal to the postmenarcheal sample. As expected (Dorgan et al., J. Natl. Cancer Inst., 95, 132-141. 2003; Groschl et al., Fertil. Steril., 76, 615-617.2001), progesterone increased following menarche, when progesterone secretion from the corpus luteum began to contribute to higher overall concentrations. Testosterone, and DHEA concentrations also demonstrated the expected increases from the premenarcheal to the postmenarcheal sample (Dorgan et al., J. Natl. Cancer Inst., 95, 132-141. 2003; Ankarberg et al., J. Clin. Endocrinol. Metab., 84, 975-984. 1999; Granger et al., Horm. Behav., 35: 18-27, 1999; Mitamura et al., J. Clin. Endocrinol. Metab., 85, 1074-1080. 2000).

Progesterone also fell into a different group than testosterone and DHEA relative to a priori predictions about the dynamic index. From the premenarcheal to the postmenarcheal samples, progesterone was expected to establish more stable, long-term changes in each hormone as the ovarian follicle develops and as the corpus luteum is differentiated following ovulation. The postmenarcheal dynamic index for progesterone is therefore a measure of the amplitude of the change in progesterone from the follicular to the luteal phase whereas premenarcheal progesterone dynamic index reflects patterns of day-to-day variability that are not yet organized into the longer-term pattern of a menstrual cycle. Thus, the temporal pattern of variability contributing to the progesterone dynamic index before menarche is not biologically equivalent to the temporal pattern contributing to the dynamic index after menarche. The population was, therefore, stratified by menarcheal status for progesterone analysis.

In contrast, the dynamic index for testosterone and DHEA has no a priori directional prediction about changes from the premenarcheal to the postmenarcheal sample. Nevertheless, diurnal rhythms for both hormones are changing during puberty (van Hooff et al., J. Clin. Endocrinol. Metab., 85, 1394-1400. 2000; Ankarberg et al., J. Clin. Endocrinol. Metab., 84, 975-984. 1999; Mitamura et al., J. Clin. Endocrinol. Metab., 85, 1074-1080. 2000), so the population was stratified by menarcheal status for androgen analysis.

Stepwise linear regression models were used to assess the predictors of hormone concentration and dynamic index. Models were mixed, combined with P <0.05 to enter and P <0.05 to remain in the model. Sexual maturation variables (age, age², and age³ plus categorical variables to represent the nominal scoring for breast development and pubic hair distribution) were deemed significant at this alpha level. Criteria for significant effects of anthropometry and lifestyle variables were more stringent with entry into the model permitted at P ≦0.05, but significance determined by P ≦0.005 in the final model after all variables with P ≦0.05 were entered. In other words, the final model incorporates all predictor variables that met the criterion of alpha ≦0.05 but associations with lifestyle variables linked to breast cancer are only discussed as significant results if the association could have occurred by chance only 5 times in 1000. This differential was based on a priori levels of biological plausibility. Association between sexual maturation stage and either the concentration or the dynamic index was intrinsically plausible because of the known role of each in the pubertal transition. In contrast, the a priori plausibility for associations between lifestyle variables linked to breast cancer and the concentration or dynamic index was not based on previously established relationships. The dynamic index has never been used as a dependent variable before, nor has it been linked directionally to breast cancer risk. Thus, it is not possible to make an a priori directional prediction about the association between lifestyle variables and the dynamic index. Risk could be associated with a high index or a low one. Risk could be associated with at a high index for one hormone and a low index for another. To avoid spurious detection of association at this low sample size, we therefore imposed the stricter criteria.

The adolescent years are a period of rapid skeletal growth and attainment of adult height. Thus, height was corrected for age with a cubic fit across all 56 participants and the residuals were used in subsequent analyses.

All statistical analyses were performed using JMP version 5.0.1a (SAS Institute Inc., Cary, N.C.).

Results

Maturational Changes

Stratification of participants by menarcheal status revealed expected patterns in the salivary hormone data (Table 3). Mean concentration of three of the four hormones (progesterone, testosterone, and DHEA) was significantly higher in the postmenarcheal than the premenarcheal sample.

TABLE 3
Descriptive characteristics of the sample stratified by menarcheal status.

| | Premenarcheal (N = 22) | | Postmenarcheal (N = 34) | | |
|---|---|---|---|---|---|
| | Mean | SD | Mean | SD | P value |
| Sexual Maturation | | | | | |
| Age (y) | 11.8 | 1.5 | 14.9 | 1.5 | <0.0001 |
| Breast Development | 2.0 | 1.2 | 3.9 | 0.9 | <0.0001 |
| Pubic Hair | 2.1 | 1.0 | 3.7 | 0.7 | <0.0001 |
| Anthropometry | | | | | |
| Height (m) | 1.51 | 0.13 | 1.66 | 0.08 | <0.0001 |
| Body mass Index (BMI) | 17.5 | 2.0 | 20.9 | 2.3 | <0.0001 |
| Body Fat (%) | 17.3 | 6.3 | 25.4 | 5.6 | <0.0001 |
| Lifestyle | | | | | |
| Caloric Intake (kcal/day) | 1904 | 627 | 1817 | 572 | 0.60 |
| Dietary Animal Fat (%) | 16.1 | 4.3 | 14.7 | 3.6 | 0.21 |
| Physical Activity (h/wk) | 30.0 | 18.3 | 20.1 | 12.1 | 0.03 |
| Physical Inactivity (h/wk) | 8.6 | 6.7 | 8.1 | 5.9 | 0.76 |
| Hormone Concentration | | | | | |
| Estradiol (pg/ml) | 4.8 | 1.5 | 5.2 | 2.4 | 0.44 |
| Progesterone (pg/ml) | 37.9 | 23.7 | 88.0 | 59.5 | <0.0001 |
| Testosterone (pg/ml) | 33.0 | 21.4 | 57.1 | 25.4 | 0.0009 |
| DHEA (pg/ml) | 36.6 | 20.0 | 100.8 | 55.3 | <0.0001 |
| Hormone Variability | | | | | |
| Estradiol dynamic index | 41.5 | 21.3 | 47.1 | 25.1 | 0.37 |
| Progesterone dynamic index | 52.7 | 15.8 | 66.3 | 18.8 | 0.007 |
| Testosterone dynamic index | 35.7 | 9.6 | 34.5 | 10.0 | 0.67 |
| DHEA dynamic index | 34.6 | 7.5 | 40.6 | 11.9 | 0.03 |

The dynamic hormone index was considerably larger than the intra-assay measurement error, ranging from a low of 34.5% (SD 10) to a high of 66.3% (SD 18.8) (Table 3). As expected, the establishment of a menstrual cycle that included expected times of low concentration and of higher concentration significantly increased the dynamic index for progesterone. Testosterone was not expected to establish a menstrual pattern, and the dynamic index for testosterone did not differ by menarcheal status. A modest, but statistically significant, increase in DHEA dynamic index from premenarcheal to postmenarcheal samples was also seen. The extent of inter-individual differences in dynamic index was therefore sufficient to support further analyses of this biomarker relative to the anthropometric and lifestyle variables.

Postmenarcheal girls had a greater height, BMI, and % body fat than premenarcheal girls. Of the lifestyle variables, only levels of vigorous physical activity were lower in the postmenarcheal than in the premenarcheal group. In contrast to the intuitive inverse association between the measure of physical activity and the measure of physical inactivity, there were significant positive associations in the sample (P <0.0005, R²=0.21), and in the sample stratified by menarcheal status (premenarcheal P <0.02, R2=0.22; postmenarcheal P=0.005, R²=0.19). Age-corrected measures of physical activity retained the same positive associations in the sample (P <0.0005, R=0.19), and in the sample stratified by menarcheal status (premenarcheal P=0.02, R²=0.20; postmenarcheal P=0.01, R²=0.17). Both measures were retained in statistical models.

Associations Between Hormone Concentration and Hormone Variability

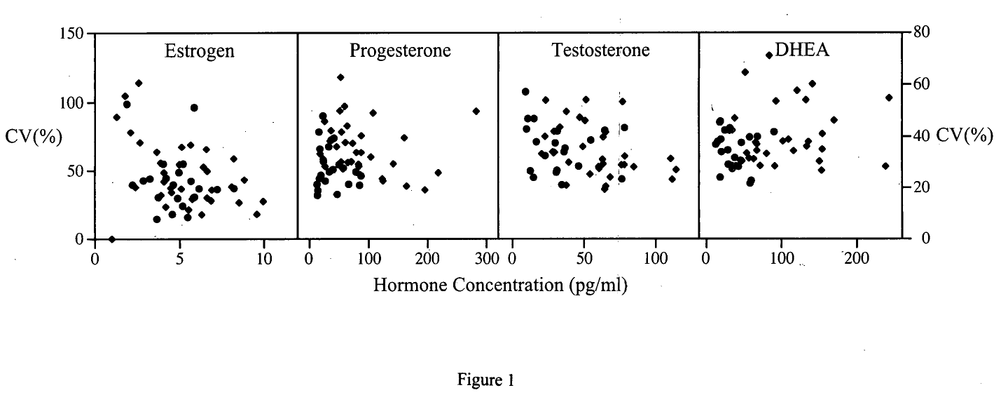

The dynamic hormone index and the hormone mean were largely independent (FIG. 1). For progesterone, there was no evidence of association across all 56 participants (P=1.0), and no evidence for association within reproductive status (premenarcheal P=0.41; postmenarcheal P=0.24). For testosterone, there was a weak negative association (P <0.02, R²=0.08) in the postmenarcheal (P <0.04, R=0.11) but not the premenarcheal sample (P=0.42). There was no association for DHEA (overall P=0.17; premenarcheal P=0.58; postmenarcheal P=0.77). In contrast, low estradiol concentration was associated with high dynamic index. After exclusion of one individual with a dynamic index of zero (17 saliva samples, all below the detection limit of the assay, and all rounded to 1.0 pg/ml), the overall association was negative (P <0.001, R²=0.19). As low estradiol concentrations were subject to the highest measurement error (Table 2), no analysis of estradiol dynamic index relative to sexual maturation and lifestyle variables was conducted.

Associations Between Lifestyle Variables and Hormone Concentrations

After stratification by menarcheal status, age continued to be a predictor of hormone concentration in both pre- and postmenarcheal models. For premenarcheal testosterone and DHEA concentrations, age³ (cubic fit where y=a(x)³+b(x)²+cx+d; x=age) was the first variable in the model. For postmenarcheal progesterone and DHEA concentration models, age was significant as a linear variable. In the two premenarcheal models where age was not a significant predictor of hormone concentration, either breast development or pubic hair distribution represented sexual maturation stage in the model.

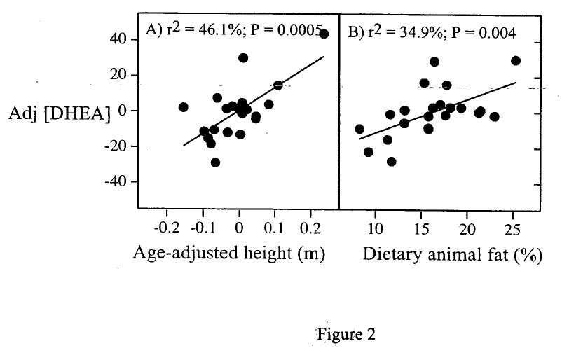

Of the three anthropometric variables, only age-adjusted height was a significant predictor of a hormone concentration. The association was positive, with individuals of above average height for age having higher DHEA concentrations (FIG. 2a). BMI and % body fat met the criteria for inclusion in the premenarcheal and postmenarcheal testosterone concentration models, respectively.

Only one lifestyle variable, the percentage of daily caloric intake derived from animal fat, met the criteria for inclusion. After correction for age³ and age-adjusted height, there was a positive association between DHEA concentration and dietary animal fat (FIG. 2b). Both physical activity and inactivity explained additional variance in the premenarcheal DHEA concentration model. Those variables increased the positive association but did not meet the criteria for independent significance. The fourth lifestyle variable, total caloric intake met criteria for inclusion in the postmenarcheal DHEA model but did not meet significance criteria. Thus, each anthropometric and lifestyle variable was able to explain a significant proportion of the hormone concentration variance in at least one model. Two of the variables met the more stringent criteria for significance. Both significant associations were positive.

TABLE 4

| | | | Anthropometry | | | | Lifestyle | | | |
|---|---|---|---|---|---|---|---|---|---|---|
| Sexual Maturation | | | | | % Body | Caloric | % Animal | | In- | Model |
| Age† | Breast† | Pubic Hair†† | Height††† | BMI | Fat | Intake | Fat | Activity | activity | R² |

Hormone Concentration

| | | | | | | | | | | | |
|---|---|---|---|---|---|---|---|---|---|---|---|
| Premenarcheal (N = 22) | | | | | | | | | | | |
| Estrogen (pg/ml) | | ***33.2+ | | | | | | | | | 33.2 |
| Progesterone (pg/ml) | | | *26.4+ | | | | | | | | 26.4 |
| Testosterone (pg/ml) | ***50.9+ | | | | | **17.5+ | | | | | 68.5 |
| DHEA (pg/ml) | ****31.5+ | | | ****20.2+ | | | | ****19.0+ | *7.0+ | **6.5− | 83.8 |
| Postmenarcheal (N = 34) | | | | | | | | | | | |
| Estrogen (pg/ml) | | | | | | | | | | | 0 |
| Progesterone (pg/ml) | ****31.9+ | | | | | | | | | | 31.9 |
| Testosterone (pg/ml) | | | | | *12.7− | | | | | | 12.7 |
| DHEA (pg/ml) | ****31.2+ | | | | | | *11.1− | | | | 42.4 |

Hormone Variability

| | | | | | | | |
|---|---|---|---|---|---|---|---|
| Premenarcheal (N = 22) | | | | | | | |
| Progesterone | | | | | | ****50.1− | 50.1 |
| dynamic Index | | | | | | | |
| Testosterone | | ***26.2− | | ***34.7+ | | | 60.9 |
| dymanic index | | | | | | | |
| DHEA | | *18.6− | | | | | 18.6 |
| dynamic index | | | | | | | |
| Postmenarcheal (N = 34) | | | | | | | |
| Progesterone | | | **17.8+ | | *10.8+ | | 28.6 |
| dynamic Index | | | | | | | |
| Testosterone | | | | | | | 0 |
| dymanic index | | | | | | | |
| DHEA | **25.9+ | *9.7− | | | | | 35.6 |
| dynamic index | | | | | | | |

*P ≦ 0.05;
**P ≦ 0.01;
***P ≦ 0.05;
****P ≦ 0.001;
P value for variable in final model
Values R² upon entering the model
Boldface Significant effects with alpha ≦0.05 for sexual maturation and alpha ≦0.005 for anthropometry and lifestyle variables
+/− Positive or inverse association in the model
† Linear and/or quadratic and/or cubic estimates of age were allowed to enter models
†† One or more dummy variables comparing the nominal stages
††† Cubic adjustment for age across all 56 participants applied before analysis

Associations Between Lifestyle Variables and Hormone Variability

Sexual maturation variables were included in dynamic index models although age met criteria for significance in only one of six models, where it was a positive association. Pubic hair distribution explained variance in three models, all of which were inverse associations. Breast development did not meet criteria for inclusion in any dynamic index models.

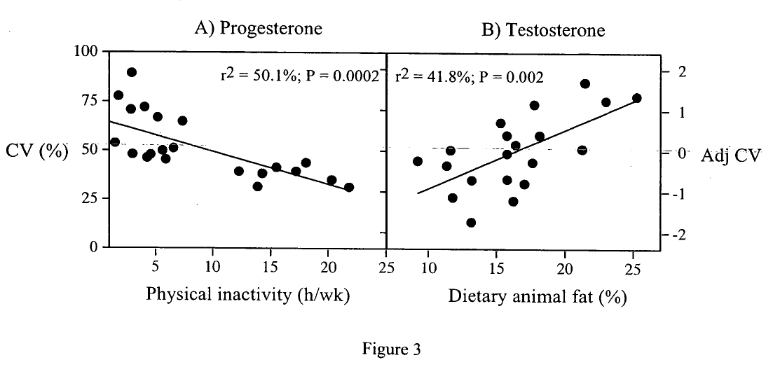

None of the anthropometry variables met criteria for inclusion in dynamic index models. In premenarcheal models, a significant inverse association between physical inactivity and progesterone dynamic index accounted for 50% of the variance (FIG. 3a). Dietary animal fat intake also had a significant positive association with premenarcheal testosterone dynamic index. An inverse association with a variable representing pubic hair distribution was also included. The relationship between dietary animal fat intake and premenarcheal testosterone dynamic index, standardized by pubic hair distribution, is shown in FIG. 3b. Two lifestyle variables, total caloric intake and physical activity, each met criteria for inclusion in the postmenarcheal progesterone dynamic index model, however the effects in the final model did not meet significance criteria.

Discussion

This study was designed in the context of a hypothesis that modification of the extent of individual variability in hormone concentration over time was a plausible biological mechanism through which lifestyle could alter sex steroid hormone exposure, and thereby alter breast cancer risk. To the best of our knowledge, it represents the first time that a measure of intra-individual hormone variability over time (dynamic index) has been proposed as an etiologic factor contributing to breast cancer risk. In spite of the small sample size, and the intrinsic developmental changes in sex steroid dynamics through time in adolescent girls, the dynamic index was associated with two of the four adolescent risk factors for breast cancer considered in this study.

The first objective of the study was to quantify day-to-day hormone variability through calculation of a dynamic index and to determine the range of variability among adolescent girls. The dynamic hormone index was large, with dynamic index for the progesterone, testosterone, and DHEA ranging from 34 to 66% across the premearcheal and postmenarcheal samples. As expected, progesterone dynamic index was the largest, and increased after menarche in response to secretion of progesterone by the corpus luteum of a menstrual cycle. Although age reliably predicted progesterone concentration, it did not predict progesterone dynamic index in premenarcheal or postmenarcheal girls, in spite of the expectation that luteal insufficiency rates from failure to ovulate would decline with age in the postmenarcheal sample (Apter et al., J. Clin. Endocrinol. Metab., 57: 82-86, 1983; Reading et al., Int. J. Sport Nutr. Exercise Metab., 12: 93-104, 2002; De Souza et al., J. Clin. Endocrinol. Metab., 88: 337-346, 2003). About 40% of cycles in the year following menarche and 20% of cycles three years after menarche are expected to be anovulatory (Apter et al., J. Clin. Endocrinol. Metab., 57: 82-86, 1983). Progesterone dynamic index also varied across individuals, with the standard deviation of the sample dynamic index approximately 30% of the mean dynamic index in both the premenarcheal and postmenarcheal samples. Androgen dynamic index was also large, ranging from 34 to 41%, and varied among individuals, with the standard deviation of the sample dynamic index equal to 22-29% of the mean dynamic index. Androgen dynamic index did not have an a priori expectation of directional change with menarcheal status, and the only change seen was an increase in the dynamic index from the premenarcheal to the postmenarcheal sample. Thus, the population distribution for dynamic hormone index demonstrated substantial inter-individual variability. As such, the dynamic index has the potential to array individuals along a continuum of day-to-day hormone variability, allowing the dynamic index to be tested as a dependent variable in epidemiological studies.

The second objective was to explore possible relationships between the dynamic index and adolescent anthropometric and lifestyle variables linked to breast cancer risk in adulthood. With the small sample, an absence of association between the dynamic index and adolescent lifestyle variables linked to breast cancer would not have been cause to reject the hypothesis. The magnitude of observed associations, on the other hand, suggested that the underlying relationships might be unexpectedly strong.

First, models incorporating sexual maturation, anthropometry, and adolescent risk factors for breast cancer (age-adjusted height, physical activity and inactivity, and dietary animal fat intake) were used to predict hormone concentration. Each of estradiol, progesterone, testosterone, and DHEA concentration was predicted by one or more measures of sexual maturation. Only DHEA concentration was predicted by adolescent risk factors for breast cancer. Premenarcheal DHEA concentration was predicted by age-corrected height and percent of caloric intake derived from dietary animal fat with positive associations. Although no consistent relationship between adult height and breast cancer risk has been established, adolescent tallness (Romundstad et al., Int. J. Cancer, 105: 400-403, 2003; Hilakivi-Clarke et al., Br. J. Cancer, 85: 1680-1684, 2001), early attainment of adult height (38), and proportionate leg length (Lawlor et al., British Journal of Cancer, 89: 81-87, 2003) are breast cancer risk factors (Okasha et al., Breast Cancer Res. Treat., 78: 223-276, 2003) that might be a proxy for other dietary or hormonal exposures and/or a reflection of perinatal anthropometric variables (Romundstad et al., Int. J. Cancer, 105: 400-403, 2003; Lawlor et al., British Journal of Cancer, 89: 81-87, 2003; Chellakooty et al., J. Clin. Endocrinol. Metab., 88, 3515-3520. 2003). An age-related increase in DHEA within the premenarcheal sample was predicted by adrenarche, as DHEA is primarily produced by the adrenal gland (Grumbach et al., In: V. H. T. James, M. Serio, G. Giusti, and L. Martini (eds.), The Endocrine Function of the Human Adrenal Cortex, pp. 583-616. New York: Academic Press, 1979). Age-adjusted height was a significant predictor of age-adjusted salivary DHEA concentration. Studies of hormone concentrations in premenarcheal girls with associated individual anthropometry are rare, but premenarcheal DHEA might directly affect adult height (van Hooff et al., J. Clin. Endocrinol. Metab., 85, 1394-1400. 2000; Ghirri et al., Gyn. Endocrinol., 15, 91-97. 2001).

Within the same model, premenarcheal DHEA concentration was also positively associated with dietary animal fat intake. As diet is modifiable, it is an important target of primary prevention research (Stevens et al., Prevent. Med. 36, 594-600. 2003). Original interest in dietary fat as a risk factor for breast cancer derived from patterns of risk in different cultures, and changes in risk with migration (Ziegler et al., J. Natl. Cancer Inst., 85: 1819-1827, 1993; Ziegler et al., J. Natl. Cancer Inst., 88: 650-660, 1996). However, this pattern has not routinely emerged in prospective studies (Willett et al., Sem. Cancer Biol., 8: 245-253, 1998), perhaps because of methodological variability (Thomson et al., Am. J. Epidemiol., 157, 754-762. 2003; Bingham et al., Lancet 362, 212-214.2003) and the variety of food contributing different fat sources (Cho, E et al., J. Natl. Cancer Inst., 95: 1079-1085, 2003). For this study, dietary intake from animal fat, rather than total dietary fat, was the variable considered. This decision was based on a recent study in which premenopausal animal fat intake was associated with increased breast cancer risk in the Nurses' Health Study II (Cho, E et al., J. Natl. Cancer Inst., 95: 1079-1085, 2003). Dietary intervention to reduce breast cancer risk through a reduction in fat intake successfully decreases circulating levels of sex steroid hormones in premenopausal women (Boyd et al., Br. J. Cancer, 76: 127-135, 1997; Wu et al., J. Natl. Cancer Inst., 91: 529-534, 1999). In addition, an adolescent intervention to reduce serum low-density lipoprotein cholesterol in children decreased bioavailable estradiol during the follicular phase and increased luteal phase testosterone in the intervention group (Dorgan et al., J. Natl. Cancer Inst., 95, 132-141. 2003). DHEA-sulphate was not affected. That study did not report animal fat consumption although both total and saturated fat intake were effectively reduced by the intervention (Dorgan et al., J. Natl. Cancer Inst., 95, 132-141.2003). Other prospective measures of adolescent diet linked to premenopausal hormone measurements are not available for comparison.

This study linked two adolescent risk factors for breast cancer, age-adjusted adolescent height and dietary animal fat consumption, to concentrations of DHEA in saliva of premenarcheal girls. Both associations occur at a time in life when breast tissue is actively developing (Brisken et al., J. Mammary Gland Biol. Neoplasia, 7: 39-48, 2002) and breast cancer risk is likely to be influenced by lifestyle choices (Frazier et al., Breast Cancer Res., 5: R59-R64, 2003; Okasha et al., Breast Cancer Res. Treat., 78: 223-276, 2003). There is also evidence that adult DHEA plays a direct role in the stimulation of breast cell proliferation (Morris et al., Surgery, 130: 947-953, 2001), and that variant androgen receptors, with unknown differential binding affinity for testosterone and DHEA, might be protective against breast cancer (Giguére et al., Cancer Res., 61: 5869-5874, 2001). Thus, these associations with DHEA concentrations are a function of maturation of the adrenal gland and adrenarchal status, yet might also be etiologic factors in breast cancer.

Similar models incorporating sexual maturation, anthropometry, and adolescent risk factors for breast cancer (age-adjusted height, physical activity and inactivity, and dietary animal fat intake) were used to predict dynamic hormone index. Dynamic hormone index was associated with lifestyle variables. Specifically, dietary animal fat intake was positively associated with premenarcheal testosterone dynamic index and physical inactivity was inversely associated with premenarcheal progesterone dynamic index. The dietary animal fat association was independent of age, and improved by inclusion of a sexual maturation variable (pubic hair distribution), so we conclude that the association is independent of sexual maturation.

A number of recent studies have moved physical inactivity from a suspected (Bernstein et al., J. Natl. Cancer Inst., 86: 1403-1408, 1994) to a known risk factor for breast cancer (Friedenreich et al., Am. J. Epidemiol., 154: 336-347, 2001; Friedenreich et al., Epidemiology, 12: 604-612, 2001; Friedenreich et al., Med. Sci. Sports Exercise, 33: 1538-1545, 2001; Yang et al., Cancer, 97: 2565-2575, 2003). As physical activity is modifiable, and interventions early in life are most likely to have lasting effects on lifetime activity, associations between activity variables and hormone exposures are important components of primary prevention strategies (Friedenreich et al., Chronic Disease Can., 22: 41-49, 2001). In one recent study, recall of physical activity at age 16 was inversely associated with premenopausal breast cancer risk (Dorn et al., Med. Sci. Sports Exercise, 35: 278-285, 2003). Physical inactivity is also an emerging epidemic in the western world (Tremblay et al., Int. J. Obesity Related Metabol. Disorders, 26: 538-543, 2002) linked to the metabolic syndrome X and obesity (Katzmarzyk et al., J. Clin. Epidemiol, 54: 190-195, 2001), coronary heart disease risk (Katzmarzyk et al., Preventive Med., 29: 555-562, 1999), and adolescent physical condition (Katzmarzyk et al., Med. Sci. Sports Exercise, 30: 709-714, 1998). Childhood measures of physiology including childhood adiposity (Katzmarzyk et al., J. Clin. Epidemiol, 54: 190-195, 2001; Katzmarzyk et al., Amer. J. Clin. Nutr., 69: 1123-1129, 1999; Campbell et al., Obesity Res., 9: 394-400, 2001), blood pressure (Katzmarzyk et al., Prevent. Med., 31: 403-409, 2000), and blood lipids (Katzmarzyk et al., J. Clin. Epidemiol, 54: 190-195, 2001) are also more predictive of adult values than behavioral risk factors (Campbell et al., Am. J. Human Biol., 13: 190-196, 2001; Fortier et al.,. Med. Sci. Sports Exercise, 33: 1905-1911, 2001).

In the current study, levels of inactivity were inversely associated with premenarcheal progesterone dynamic index. In the absence of an effect of age or other sexual maturation variables, this is unlikely to reflect an effect of physical inactivity on the timing of sexual maturation. Given current concern about the role of combined estradiol and progestin in breast cancer etiology (Chlebowski et al., J. Am. Med. Assoc., 289: 3243-3253, 2003), and weak associations between postmenarcheal progesterone dynamic index and both caloric intake and physical activity, further studies of progesterone dynamic index and possible associations with lifestyle variables are contemplated.

The estradiol dynamic index could not be reliably calculated in this study because the contribution of methodological variability could not be separated from individual variability. With methodological refinement to pre-concentrate salivary estradiol before analysis, so that the dynamic index can be calculated without the measurement error intrinsic to low concentrations in this study, estradiol dynamic indices will be calculable.

In the current study, associations between lifestyle variables and dynamic hormone index were restricted to the premenarcheal sample. Both menstrual hormone dynamics and ovulatory status might have masked effects of lifestyle variables in the postmenarcheal sample. However, in larger postmenarcheal samples, concentration of progesterone during the luteal phase of postmenarcheal girls could be used to stratify the sample according to ovulatory status. In addition, samples could be retrospectively assigned to a phase of the menstrual cycle and dynamic index could be calculated for each phase. In addition, retrospective assignment of gynecological age to premenarcheal girls would be useful to clarify temporal relationships of dynamic index to impending menarche.

The study design has several strengths. First, the repeated measures design results in a high confidence estimate of both hormone mean and dynamic hormone index that minimizes measurement error and yields more statistical power to detect associations than an equivalent population without repeated sampling. Second, salivary samples only contain bioavailable steroid hormone, so that the dynamic hormone index reflects the variability in concentration that cells within the individual are experiencing more accurately than a dynamic index calculated from total (bound plus unbound) hormone concentrations in serum or plasma. This focus on bioavailable hormone might also increase statistical power because it bypasses potentially confounding effects of binding-protein dynamics (Key et al., J. Natl. Cancer Inst., 95, 1218-1226. 2003; McTieman et al., J. Clin. Oncol., 21: 1961-1966, 2003; Dorgan et al., J. Natl. Cancer Inst., 95, 132-141. 2003; Gann et al., J. Natl Cancer Inst., 88: 1118-1126, 1996). Third, the predictor variables entered into analyses were few in number, and were chosen based on the best available epidemiologic evidence associating adolescent variables to breast cancer risk (Cho, E et al., J. Natl. Cancer Inst., 95: 1079-1085, 2003; Okasha et al., Breast Cancer Res. Treat., 78: 223-276, 2003). Of the four adolescent risk factors, three met significance criteria in this study.

This study population, attending private school, had a higher socioeconomic status (SES) than the general population. Nevertheless, the population had expected mean caloric intake, median animal fat consumption, and BMI (Cho, E et al., J. Natl. Cancer Inst., 95: 1079-1085, 2003; Frazier et al., Breast Cancer Res., 5: R59-R64, 2003; Katzmarzyk et al., Preventive Med., 29: 555-562, 1999; Dorgan et al., J. Natl. Cancer Inst., 95, 132-141. 2003; Gray-Donald et al., Can. J. Pub, Health, 381-385, 2000), although it was less obese than the general population (Tremblay et al., Int. J. Obesity Related Metabol. Disorders, 26: 538-543, 2002). High SES is an accepted risk factor for breast cancer (Byrne, C. Risks for major cancers—breast. In: A. Harras (ed.), Cancer: rates and risks, pp. 120-123. Washington, D.C.: Department of Health and Human Service, Public Health Service, National Institutes of Health, 1996) that might restrict associations to high SES groups. On the other hand, a sample more representative of the general population might reasonably be expected to reveal stronger associations. The dynamic index is sufficiently variable that it can be used to array individuals along a population-based continuum. The immediate focus of the Daughters Without Breast Cancer research project is on assessing the stability of individual dynamic hormone index measures from one year to the next.

To our knowledge, this is the first study to examine potential associations between anthropometric and lifestyle variables previously linked to breast cancer risk and the extent of individual hormone variability. The study was based on a biologically plausible hypothesis that intra-individual measures of hormone dynamics might prove to be an etiologic factor contributing to breast cancer risk. In spite of the small scale of this study, associations between adolescent risk factors for breast cancer and steroid hormone variability (dynamic index) were present in a population of adolescent girls.

### Example 2

### Automated Measurement of Dynamic Hormone Indices

A robotic liquid handling system (e.g., Biomek FX) and compatible off-deck equipment are used to automate (salivary) hormone assays for precision and high throughput. Daily, weekly, and monthly controls are integrated into the operations of the equipment. Each day, on each hormone plate, calibration standards and high and low concentration controls are run. Weekly, pipetting precision is checked through calorimetric quantification of dye pipetted into blank wells to emulate assay methods. Monthly, or whenever batch number changes, hormone assay plates are devoted to quality controls to identify edge effects and within-plate sources of variability. Also on a monthly basis, saliva samples are spiked with tritiated hormone and put through the solid phase extraction (SPE) (e.g., C18) procedure to quantify recovery and variability in recovery following extraction. At the same time, repeated measurement and extraction of the same saliva sample are used to quantify sources of error within the method.

### Example 3

### Dynamic Hormone Index and Breast Cancer

### Measurement of Estradiol in Premenarcheal Women

An epidemiological study of 200 premenarcheal women (ages 32-36) with a strong family history of premenarcheal breast cancer (greater than or equal to 2 first-degree relatives) is compared to 200 control women matched for age, socioeconomic status, reproductive history. Saliva is collected daily during each of two menstrual cycles, separated by three months. The onset of menstrual bleeding is used to delimit the cycle. A commercial ovulation detection kit (e.g., urine kit) is used to pinpoint the day of ovulation. The luteal phase is defined as all samples from 24 h after ovulation to 24 h before the onset of the next menstrual bleeding.

The dynamic index for estradiol during the luteal phase is expected to be significantly higher (P less than or equal to 0.05 after controlling for age and reproductive history) in women with a family history of breast cancer. Closer examination of the underlying statistical distribution is expected to show that the dynamic index has a bimodal distribution in the family history group, with approximately 20 women (10%) having a dynamic index greater than 95th percentile for the control population.

### Example 4

### Dynamic Hormone Index and Breast Cancer

### Measurement of Estradiol in Women Under Treatment

An epidemiological study in luteal phase estradiol dynamic index is calculated on 2000 women (ages 15-45) and the population is partitioned into quintiles. Each quintile is divided into one of three groups: (1) oral contraceptive (combined, triphasic low dose pill) plus placebo transdermal patch; (2) placebo pill plus placebo patch; (3) transdermal estradiol patch plus triphasic progestagen only pill. The dynamic index is for each individual is calculated again during the second and third month of treatment. After two months, treatment groups are changed in a randomized-controlled, block design.

It is expected that a reduced individual luteal phase estradiol dynamic index will be observed in group 3 only, and that treatment will be most effective in the upper two quintiles. It is also expected that women with an inherent low estradiol dynamic index do not differ under the three treatments. A randomized controlled trial of transdermal estradiol patch with oral tri-phasic progestagen, to assess breast cancer relative risk of individuals, will be conducted.

### Example 5

### Dynamic Hormone Index and Diet

### Measurement of Testosterone in Hamster Model

Animal models demonstrate that the hormone dynamic index is affected by modifiable lifestyle choices. In one study, male hamsters belonging to an outbred laboratory population were challenged with a diet that matched their usual diet in all respects (fibre content, water content (10%), total calorie value, protein, etc.) except that a larger proportion of the total calories were derived from fat (Purina 5001=12% vs. test diet=25%; Table 5). All fat in the high fat diet was animal in origin (lard). When provided with a high animal fat diet, this hamster species (Phodopus sungorus) does not become obese (Bartness et al. (1985) Physiology and Behaviour. 35:805-808), so the dietary manipulation is not confounded by changes in body composition.

TABLE 5
Comparison of the two diets used

| | | Modified Laboratory Rodent Diet |
|---|---|---|
| | Laboratory Rodent Diet 5001 | with 25% Energy from Fat (Lard) |
| Chemical Composition | <http://www.labdiet.com> | <http://www.testdiet.com> |
| Crude protein not less than (%) | 23.0 | 23.9 |
| Crude Fat not less than (%) | 4.5 | 10.1 |
| Crude fiber not more than (%) | 6.0 | 5.1 |
| Ash not more than (%) | 8.0 | 6.7 |
| Calories provided by protein (%) | 28.049 | 26.325 |
| Calories provided by fat (ether extract %) | 12.137 | 25.031 |
| Carbohydrates (%) | 59.814 | 48.844 |
| Physiological Fuel value (kcal/gram) | 4.61 | 5.252 |
| Moisture content (%) | 10 | 10 |

Materials and Methods

A total of 12 males were used in a single study that lasted 12 weeks. All males remained in their home cages and were moved to a new room without other animals, but with identical environmental conditions to the previous room. Six were fed the usual diet and six the high fat diet. Except for daily weighing, animals were undisturbed for two weeks. From the 3^{rd} to the 6^{th} week, 12*150 μl volumes of blood were obtained into 75111 heparinized capillary tubes from the retro-orbital sinus of each animal, under isoflurane anesthesia, using the method detailed in Reburn and Wynne-Edwards (Comp Med. 50:184-98, 2000). Blood was collected each Monday, Wednesday and Friday at the same time of day (in the middle of the light phase of the 14 h light: 10 h dark light cycle) for each individual (±15 minutes) to preclude variability in hormone concentration resulting from underlying circadian rhythmicity (Hoffmann et al. (1977) Acta Endocrinologica 86:193-199). On the 43 day, each animal was switched to the opposite diet and the procedure was repeated with two weeks to acclimatize and four weeks of blood sampling to yield a further 12 blood samples.

Each blood sample was quantified in a commercial radioimmunoassay for free testosterone (Inter Medico of Markham, ON: Coat-A-Count Free testosterone TKTF2 as a distributor for Diagnostic Products Corporation of Los Angeles, Calif.). Free testosterone differs from total testosterone because it excludes testosterone molecules that are bound to binding globulins in the blood and cannot enter cells or interact with androgen receptors. Thus, the free testosterone assay measures the bioavailable testosterone at the time of sampling. All blood samples from a single hamster were quantified within a single assay. Measurement error, or intra-assay variability, for a pool of male hamster serum quantified in quadruplicate in each of six assays was 7.9%. 43 of the 267 free testosterone determinations (16.1%) fell below the assay sensitivity of 0.15 pg/ml. Each of the 12 hamsters had at least one low concentration on each diet. Low values were rounded up to 0.15 pg/ml before analyses.

The 24 free testosterone concentrations for each male hamster were reduced to four values before analyses: (1) mean free testosterone concentration while on the high fat diet; (2) mean free testosterone concentration while on the low fat diet; (3) free testosterone dynamic index while on the high fat diet; and (4) free testosterone dynamic index while on the low fat diet. For each diet, free testosterone dynamic index for a hamster was calculated as the individual standard deviation divided into the mean free testosterone concentration times 100. Standard deviation is slightly underestimated when the number of samples is less than 30, so each standard deviation was corrected for sample size by multiplying the calculated standard deviation times [1+(1/(4*N))]. Hormone concentrations were not normally distributed within individuals (12/12), however 10/12 individual distributions met the assumptions for normality (KSL test) after log transformation. Thus, all hormone determinations were log-transformed before calculation of the means. All statistical comparisons were paired within a hamster over the two diets.

Results

In this out-bred population of laboratory-reared hamsters, profound individual variability in hormone concentration over time was typical. The hamster with the lowest free testosterone concentration while on the standard diet (low fat) was 22× lower in mean free testosterone concentration than the hamster with the highest concentration (1.27 pg/ml vs. 28.24 pg/ml). The twelve hamsters also differed dramatically in the dynamic hormone index (84% through 207%).

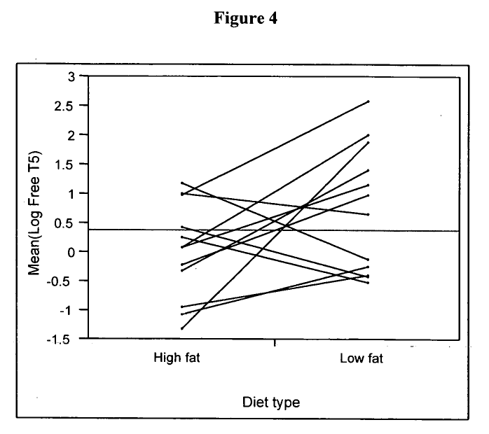

Log free testosterone concentration was not altered by the dietary manipulation. There was no effect of diet order (F(1,10)=0.09, p=0.77) or diet type (repeated measure F(1,10)=4.00, p=0.07) and no interaction of the two variables (F(1,10)=2.25, p=0.16) on log free testosterone concentration. FIG. 4 shows the mean hormone concentrations for all 12 males with lines connecting the mean concentrations for the same male on the two diets. There was no consistent pattern of increase or decrease from one diet to the other.

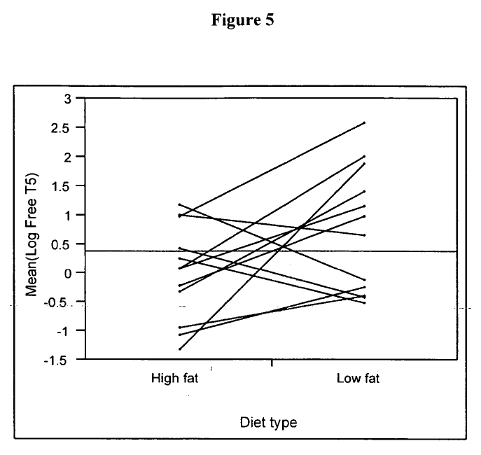

In contrast, free testosterone dynamic index was significantly increased on the high fat diet. Specifically, there was no effect of diet order (F(1,10)=1.09, p=0.32) and no interaction of diet order and diet type (F(1,10)=0.39, p=0.54), but there was an effect of diet type (repeated measure F(1,10)=5.49, p=0.04). Hormone concentration was lower on the high fat diet but the difference was not statistically significant (paired t=1.52, df=11, p=0.16). FIG. 5 shows the dynamic index for all 12 males with lines connecting the dynamic index for the same male on the two diets. Free testosterone dynamic index was higher on the high fat diet (paired t=2.42, df=11, p=0.03) and only 2/12 hamsters had a lower dynamic index on the high fat diet.

Average body weight across the last five days of the low fat diet was 92.8% of weight across the final days of the high fat diet, which was a significant change in body weight (paired t=2.56, df=10, p=0.03). Thus, it is probable that body fat also increased reversibly while eating the high fat diet.

Discussion

Wide population variability in hormone concentration is typical of human populations with the 95% reference range for free testosterone in males 20-39 years old ranging from 8.8 to 27 pg/ml (Ooi et al., (1998) Clin Biochem 31:15-21). The range of variability in the hamster population was similar at the high end, and lower at the low end. In contrast, such variability is not expected in inbred strains of rats, mice, and hamsters typically used in laboratory research.

As the dynamic hormone index for testosterone has not been calculated before, there is no basis for comparison of this result with previous literature for humans or other laboratory rodents. Nevertheless, the dynamic hormone index for individuals ranged from 84 to 207% in this small sample of hamsters. The contribution of measurement error to the dynamic hormone index was negligible (<8%). Thus, free testosterone dynamic index is a highly variable trait in this animal population.

Experimentally manipulating the animal fat content of the diet did not change hormone concentrations within individual male hamsters, but did significantly alter the dynamic index of that hormone. The observed alteration in dynamic hormone index was reversible because there was no effect of the order of diet manipulation on the dynamic hormone index. This effect is statistically significant within a small population of male hamsters in a controlled laboratory setting. The present study cannot differentiate between an effect of the high fat diet, and an effect of increasing body fat composition. These two variables are also intimately confounded in human populations. We conclude that one of the major impacts of a high animal fat diet on the physiology of an individual is likely to be a change in dynamic hormone index.

Diets high in animal fat pose numerous health risks in to humans. A high fat diet increases the risks for colon, prostate and breast cancers (Cho et al. (2003) J. Natl Cancer Instit, 95(14): 1079-85; Giovannucci et al (1993) J Natl Cancer Inst 85:1571-1579; Willett et al (1990) N Engl J Med 323:1664-1672). All of these diseases also have hormonal contributions to their etiology. Furthermore, given the directional association between high animal fat and high disease risk in human populations (Willett (1998) Sem Cancer Biol 8:245-253), we conclude that a high dynamic hormone index for free testosterone should be a valuable biomarker for increased disease risk.

### Example 6

### Dynamic Hormone Index and Exercise

### Measurement of Testosterone in Hamster Model

In this example, male hamsters belonging to an outbred laboratory population were given the opportunity for vigorous aerobic exercise by the addition of a running wheel to their cage.

Materials and Methods

A total of 12 adult males were used in a single study that lasted 12 weeks. All males were given a home cage with a larger surface area (790 vs 410 sq cm; Nalgene Animal Science product 660-1264 vs 660-1291, Fisher Scientific Ltd., Ottawa, ON), and were moved to a new room without other animals but with identical environmental conditions to the previous room. All 12 animals had a running wheel (5.5″ comfort wheel, SP-61382, Pets International Inc., Elk Grove Village, Ill.) suspended from the roof of the cage. Each wheel had three modifications: (1) a ½″ stainless steel screw and two bolts were inserted through a drilled hole in the base to provide a weight and ensure that the wheel would return to a fixed position and protect the odometer magnet (see below) from chewing by the hamster; (2) the odometer magnet (see below) was affixed to the opposite side of the wheel. It was configured with a groove intended to snap onto a bicycle spoke and a steel wire was snapped into that groove and then bent and inserted into the wheel and fixed with epoxy glue; and (3) a ¼″ hole in the back wall of the wheel was drilled to allow a 1″ stainless steel screw to be bolted into it. When inserted, that screw was tied to the suspension brace with wire so that the wheel was locked and could not turn.

In Group A, six male hamsters had the wheel locked for the first six weeks and unlocked to allow exercise for the second six weeks. In group B, animals had the opposite treatment with an unlocked wheel followed by a locked wheel. For all animals, the first two weeks were used for acclimation to the treatment, and the following four weeks were used to obtain dependent measurements. A small blood sample was obtained under isoflurane anesthesia by the method described in Example 4 on each Monday, Wednesday, and Friday for the four weeks. This yielded 12 individual blood samples with access to vigorous aerobic exercise plus 12 blood samples for the same individual when the wheel was locked and that avenue for exercise was not available.

All dependent measures were the same as in Example 4 with the addition of a daily record of distance traveled in the running wheel and daily food consumption (grams Purina 5001 rodent chow, Purina, St Louis Mo., consumed per day). Distance traveled was obtained by bicycle odometer (Sigma Sport BC500, Cyclepath, Kingston, ON) with the magnets separated by 1 cm as the wheel hung from the lid of the cage. The odometer multiplied the number of wheel rotations by the circumference of the wheel (450 mm) to display the distance traveled in kilometers per night. Food consumption was measured by drilling a ⅛th inch hole in each chow block and presenting them to the hamster stacked on a coat-hanger wire standing within a 2″ diameter, 3″ length ABS (acrylonitrile butadiene styrene) pipe with a 1.5″w×2.5″ h opening in one side. Both the base and the top were covered with an ABS cap. The lower ABS cap was covered with ¼″ hardware cloth to capture crumbs. Silicone caulking secured the coat hanger wire. All materials were purchased from a local hardware store. The entire food dispenser was weighed daily to determine food consumption.

Each blood sample was quantified in the same commercial radioimmunoassay for free testosterone (Inter Medico of Markham, ON: Coat-A-Count Free testosterone TKTF2 as a distributor for Diagnostic Products Corporation of Los Angeles, Calif.) as described in Example 4. Measurement error, or intra-assay variability, for a pool of male hamster serum quantified in quadruplicate in each of six assays was 6.1%. Fourteen of the 263 free testosterone determinations (5.3%) fell below the assay sensitivity of 0.15 pg/ml. Low values were rounded up to 0.15 pg/ml before analyses.

The 24 free testosterone concentrations for each male hamster were reduced to four values before analyses: (1) mean free testosterone concentration without access to exercise; (2) mean free testosterone concentration with access to exercise; (3) free testosterone dynamic index without access to exercise; and (4) free testosterone dynamic index with access to exercise. For each exercise regime, free testosterone dynamic index for a hamster was calculated as his individual standard deviation divided into his mean free testosterone concentration, times 100. Standard deviation is slightly underestimated when the number of samples is less than 30, so each standard deviation was corrected for sample size by multiplying the calculated standard deviation times [1+(1/(4*N))]. To satisfy the statistical requirement for a normal distribution in the hormone concentrations, each free testosterone determination was log-transformed before calculation of the mean. All statistical comparisons were paired within a hamster over the two exercise regimes and considered the order of the regimes (wheel locked first or unlocked first) as a variable in analyses.

Results

Males were highly variable in their average hormone concentration (range 2.5 to 94.2 pg/ml) and in their dynamic hormone indices (range 37.9 to 164% of the mean concentration for that male). There was no association between the concentration and the dynamic hormone index for an individual across all 24 blood samples (F(1,11)=1.17, P=0.30) or in the subsets without (P=0.15) or with (P=0.06) exercise. All animals exercised when they had the opportunity. Average distance traveled ranged from 8.9 to 22.8 km per night. There was no effect of access to the running wheel on body weight over the last five days in the treatment (paired t=0.88, P=0.41). Exercise, however, clearly required more energy. Within-male food consumption during the last five days of each treatment was 50% higher during exercise (4.46 vs. 6.71 g/day: Paired t=8.11, P <0.0001).

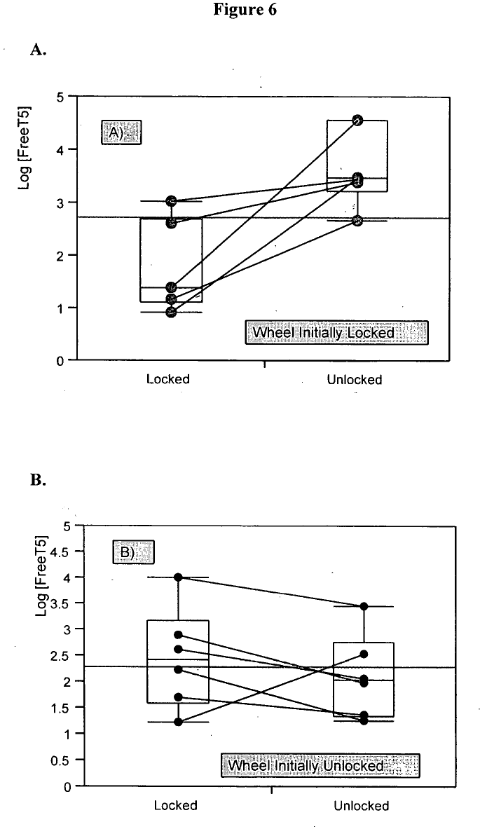

The highest free testosterone concentrations were all associated with group A in which there was access to exercise during the second six weeks. Average hormone concentration had a significant interaction between the status of the wheel (unlocked versus locked) and the treatment order (unlocked first or locked first: F(1,23)=10.7, P <0.005). When the wheel was available during the second part of the experiment, the average hormone concentration within males more than doubled with access to the wheel (Log free testosterone concentration: Paired t=3.97, df=5, P=0.01: FIG. 6A). In Group B, there was no corresponding decrease in male testosterone concentration when the wheel was locked after the exercise (Log free testosterone concentration: Paired t=0.95, df=5, P=39: FIG. 6B). Free testosterone concentration in males with the wheel initially locked, during the time the wheel was locked, was similar to the free testosterone concentration of males in Example 4 while on the low fat, standard husbandry diet.

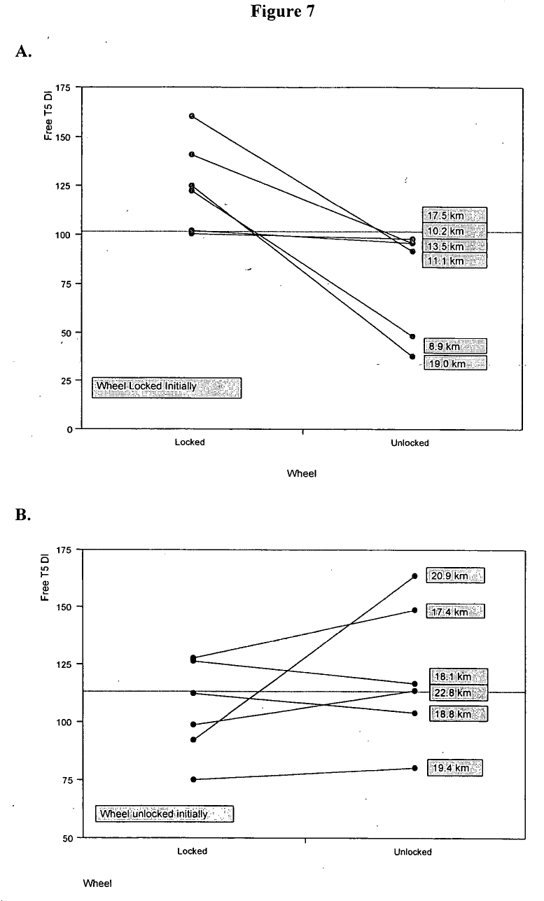

Free testosterone dynamic index also had a significant interaction between the status of the wheel (unlocked versus locked) and the treatment order (locked first or unlocked first: F(1,23)=10.4, P <0.01). Dynamic hormone index for free testosterone for individual hamsters in Group A that spent the first six weeks with a locked running wheel and the second six weeks with the opportunity to run decreased an average of 47% when the exercise was available (Paired t=−3.22, P=0.024: FIG. 7A). These low dynamic hormone index values correspond with the elevated free testosterone concentration in FIG. 6A. Dynamic hormone index for free testosterone for individual hamsters in Group B that spent the first six weeks with an unlocked running wheel and the second six weeks without the opportunity to exercise did not recover to the same values as the Group A. Individual dynamic hormone index was not lower when the wheel was available (Paired t=−1.29, P=0.25: FIG. 7B). Free testosterone dynamic index without exercise was within the range of the dynamic index range for animals on the same diet in Example 4.

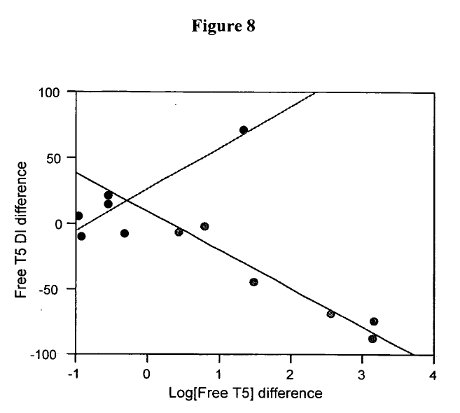

The change in log free testosterone concentration (Log[Free T5] difference: unlocked-locked) and the change in free testosterone dynamic index (Free T5 DI difference: unlocked-locked) was also affected by treatment order (FIG. 8). If the wheel was initially locked (Group A: pale dots), there was a strong negative relationship (F1,5=69.8, P=0.001: adjusted r-squared=0.93); whereas, if the wheel was initially unlocked (Group B: black dots), the relationship was positive (F1,5=15.5, P=0.02: adjusted r-squared=0.74).

Discussion

Free testosterone concentration varied widely between males, but was consistent with Example 4. Dynamic hormone index for free testosterone was also consistent with Example 4. The contribution of measurement error to the dynamic hormone index was negligible (6.1%). Thus free testosterone dynamic index was a highly variable trait in this example.

In Group A, the concentration and dynamic hormone index were first calculated while the running wheel was locked, and then calculated again after the opportunity for exercise was introduced by unlocking the wheel. Free testosterone concentration was elevated within males while exercising. Dynamic hormone index was decreased in the same males. This relationship was proportionate, with larger exercise-induced increases in testosterone concentration associated with larger exercise-induced decreases in free testosterone dynamic index.

In Group B, exercise was available initially, but then denied to the hamsters. This resulted in a very different pattern of results. There was no change in free testosterone concentration associated with exercise and there was no change in free testosterone dynamic index associated with exercise. There was, however, a positive association between the change in free testosterone concentration and the change in dynamic hormone index and this pattern was opposite to Group A. The differences between groups A and B suggest that there was an effect of vigorous aerobic exercise on hormone concentration and dynamic hormone index, but that the effect was not reversible over the time frame of this experiment. Although not quantified, it was clear that males denied access to exercise after having had the wheels continued to devote considerable energy to trying to get the wheels to move.

The persistent benefits of vigorous aerobic exercise for individual health are undisputed. Cardiovascular disease, high blood pressure, and high cholesterol (Mosca et al (2004) Arterio. Thromb & Vas Biol. 24:e29-50; Chobanian et al (2003) J Amer Med Assoc 289: 2560-2572; Expert panel (2002) Circulation 106: 3143-3421) are reduced by exercise. Links to cancer prevention are also emerging and ranked as convincing for breast and colon cancers, probable for prostate cancer, and possible or insufficient data for cancers at other sites (Friedenreich et al. (2002) J Nutr. 32(11 Suppl):3456S-3464S). For example, increased exercise at any stage of the lifespan results in a decrease in the risk of breast cancer and vigorous aerobic exercise is more effective than other forms of physical activity (Friedenreich et al (2001) Epidem. 12:604-12; Friedenreich et al. (2001) Med Sci in Spts & Exer 33:153845). For prostate cancer, risk is also reduced by vigorous aerobic exercise and exercise early in life (Friedenreich et al (2004) Am J Epid 159:740-9). A role for total or free testosterone in prostate cancer epidemiology is disputed (Stattin et al (2004) Intl J Cancer 108:418-24; Chen et al (2003) Cancer Epid Bio Prev. 12:1410-6). However, epidemiological studies attempting to link testosterone to prostate cancer have all been based on cross-sectional analyses from a single blood sample. Thus, although these results do not provide a direct link to disease incidence or outcome, they suggest that alterations in the free testosterone dynamic index is a mechanism through which exercise affects health.

### Example 7

### Dynamic Hormone Index Variability within Adult Populations

### Measurement of Testosterone in Expectant/New Parents

Two published studies, hormone concentrations were measured from saliva samples collected from men and women becoming parents for the first time and from control men that were not fathers (Berg et al. (2001) Mayo Clin. Proc. 76:582-592; Berg et al. (2002) Horm. Behav. 42:424-436). In the following Example, dynamic hormone indices were calculated for the first time using these published hormone values to quantify the range of population variability for the testosterone and cortisol dynamic hormone indices.

Method

In the published studies (Berg et al. 2001, 2002, supra), fourteen control men were distributed relative to the time of day that they collected samples. Seven of those men provided at least 11 saliva samples (range 11-19 samples) during the morning hours (0:631 through 11:00 h) and were used to calculate mean free testosterone and cortisol concentrations. In the present Example, these hormone concentrations were then used to calculate dynamic hormone indices.

In the course of these previous studies, (Berg et al. 2001 and 2002, supra), 23 men provided saliva samples over the interval from the start of the second trimester of their partner's first pregnancy until their infant was four months old. Only four of these expectant/new fathers collected their samples in the morning, but 12 expectant/new fathers consistently collected samples during the evening hours (17:00 through 23:59 h). In the evening, cortisol is at lower concentration than in the morning, because there is a post-awakening increase in cortisol (Berg et al., 2001, supra). Thus, mean free cortisol in twelve expectant/new fathers ranged from 0.03 to 0.16 μg/dl. At these low concentrations, the performance of the cortisol assay was not specifically quantified. However, we expected assay measurement error to make a substantial contribution to the cortisol dynamic index. Thus, the dynamic index for cortisol could not be calculated and only the dynamic index for testosterone was calculated for these men in the present study.

In the previous studies (Berg et al. 2001, 2002, supra), eight expectant/new mothers partnered with the expectant new fathers also provided frequent samples during the evening hours, and similar calculations were made for these expectant/new mothers.

Each saliva sample was quantified in a commercial radioimmunoassay for testosterone or cortisol (Inter Medico of Markham, ON: Coat-A-Count; Diagnostic Products Corporation of Los Angeles, Calif.). Details of the assay procedure and the assay measurement variability have been previously described (Berg et al. 2001, supra).

All hormone concentrations for each individual were reduced to four values before analyses: (1) mean free testosterone concentration; (2) mean free cortisol concentration; (3) free testosterone dynamic index; and (4) free cortisol dynamic index. The dynamic hormone index for an individual was calculated as the individual standard deviation divided into the mean free hormone concentration, times 100. Standard deviation is slightly underestimated when the number of samples is less than 30, so each standard deviation was corrected for sample size by multiplying the calculated standard deviation times [1+(1/(4*N))].

Results

Control men: Mean free testosterone concentration in the seven men ranged from 12.2 through 22.4 ng/dl. Of those seven men, six had cortisol measurements. Mean free cortisol ranged from 0.38 to 0.91 μg/dl. The dynamic hormone index was calculated for each hormone. For free testosterone the range across the seven men was from 16.9 to 52.6%. For free cortisol, the dynamic hormone index ranged from 27.1 through 75.6% across the six men.

As expected, there was no association between mean hormone concentration and dynamic hormone index for testosterone (r-squared adjusted <−0.005, P=0.37) or cortisol (r-squared adjusted=−0.13, P=0.56). Across the age range in the sample (21.9 through 46.3 years), there was no effect of age on mean free testosterone concentration (r-squared adjusted=0.09, P=0.26), mean free cortisol concentration ((r-squared adjusted=0.33, P=0.14), free testosterone dynamic index (r-squared adjusted=−0.04, P=0.42), or free cortisol dynamic index ((r-squared adjusted=−0.21, P=0.75). There was no association between the dynamic hormone indices for the two hormones (r-squared adjusted <0.001, P=0.98).

Expectant and new fathers: All samples were included in this analysis except those from the immediate interval surrounding the birth with labour and delivery (Berg et al, 2001 and 2002, supra). Twelve expectant/new fathers provided between 11 and 53 saliva samples (average=28 samples). Mean free testosterone concentration in those 12 men ranged from 8.1 through 34.7 ng/dl. The free testosterone dynamic index ranged from 1-9.0 to 51.6%. As expected, there was no association between mean free testosterone concentration and dynamic hormone index (r-squared adjusted <0.05, P=0.24). Across the age range in the sample (25.3 through 37.0 years), there was no effect of age on mean free testosterone concentration (r-squared adjusted=−0.07, P=0.62) or free testosterone dynamic index (r-squared adjusted=−0.04, P=0.48).

Expectant/new mothers: All samples were included in this analysis except those from the immediate interval surrounding the birth with labour and delivery (Berg et al. 2001 and 2002, supra). Eight expectant/new mothers provided between 18 and 52 samples each. Mean free testosterone concentration in the expectant/new mothers ranged from 1.3 through 8.5 ng/dl. At that level, the intra-assay variability is 15.9% (Berg et al., 2001, supra). This is double the measurement error allowed to contribute to the dynamic hormone index calculation in previous examples and will have contributed to the dynamic hormone index. Nevertheless, the free testosterone dynamic index was considerably larger than the measurement error and ranged from 51 to 103%. As for the fathers, the mean free cortisol concentrations (0.10 through 0.27 μg/dl) in expectant/new mothers were too low to allow accurate calculation of a dynamic hormone index for cortisol. As expected, there was no association between mean free testosterone concentration and dynamic hormone index (r-squared adjusted=0.03, P=0.32). Across the age range in the sample (27.1 through 36.3 years), there was no effect of age on mean free testosterone concentration (r-squared adjusted=−0.05, P=0.44) or free testosterone dynamic index (r-squared adjusted=0.32, P=0.08).

Comparisons: Free testosterone dynamic indices for control men and expectant/new fathers are shown in FIG. 8. Free testosterone dynamic index differed across the three groups (ANOVA F2,26=37.9, P <0.001). In this analysis, with morning samples for the control men and evening samples for the expectant/new fathers, there was no difference in mean free testosterone concentration (t=1.72, df=17, P=0.10) and no difference in free testosterone dynamic index (t=0.56, df=17, P=0.58) for the two groups of men. However, the expectant/new mothers had a significantly higher testosterone dynamic index than the men (post hoc Tukey HSD, P <0.05).

Discussion

As for previous examples, these results were for saliva samples and thus represent the free hormone, or the hormone that is biologically available to cells because it is not bound to a carrier globulin. Free testosterone dynamic index was not different in the control men and the men becoming fathers and, consistent with previous examples, the dynamic hormone index varied three-fold across individuals. The dynamic hormone index for these men, however, was lower than the free testosterone dynamic index calculated for male hamsters in Examples 4 and 5, remaining below 60%.

Free testosterone dynamic index for women, on the other hand was higher than for men. The increased measurement error for free testosterone in the expectant/new mothers will have contributed to this difference. In addition, free testosterone in expectant women increases throughout pregnancy and then decreases dramatically after the birth when the primary source of testosterone, the placenta, is delivered (Berg et al. 2002, supra). These, systematic changes in hormone concentration driven by the endocrinology of pregnancy, delivery, and postpartum nursing will have increased the standard deviation for free testosterone in these women relative to the expectations for women not becoming mothers. As the standard deviation is an essential component of the dynamic hormone index calculation, values for the general population of adult women are expected to be lower than those shown here. Nevertheless, all of these women provided samples across the pregnancy and birth and the range of dynamic hormone indices still encompassed a two-fold range across women. Thus, the free testosterone dynamic index in adult women is likely to have substantial population variability.

Dynamic index for free cortisol could only be calculated for the control men who provided samples in the morning, because the evening samples for expectant/new parents had free cortisol concentrations that were low enough to introduce substantial measurement error and preclude a dynamic hormone index calculation. In those control men, the range of free cortisol dynamic indices covered a three-fold range.

As preliminary calculations of the dynamic hormone indices for free testosterone and free cortisol in adult humans, these results suggest that men and women routinely differ widely in their dynamic hormone index. Specifically, because the range of dynamic hormone indices for free testosterone covered a three-fold range in control men, expectant/new fathers, and expectant/new mothers, it is probable that one individual might have a dynamic hormone index up to three times higher than another individual.

Hormone-responsive tissues and cells experiencing frequent doubling (100% increase) and halving (100% decrease) of bioavailable hormone concentrations are expected to be in different physiological states than tissues and cells that only experience changes of 10 or 20% over the same time. These differences are therefore biologically plausible contributors to altered individual disease risk.

### Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

1. A method for identifying a subject at risk of developing a hormone-associated condition using hormone dynamics as a biomarker comprising,
determining a dynamic hormone index of at least one hormone in the subject,
wherein an aberrant dynamic hormone index of at least one hormone in the subject when compared to the statistical distribution of the dynamic hormone index for a population is indicative that the subject is at risk of developing a hormone-associated condition.

2-3. (canceled)

4. The method of claim 1, wherein the subject is a human.

5. The method of claim 1, wherein the hormone-associated condition is cancer.

6. The method of claim 5, wherein the cancer is selected from the group consisting of breast cancer, ovarian cancer, uterine cancer, cervical cancer, prostate cancer, testicular cancer, thyroid cancer, parathyroid cancer, adrenal gland cancer, colon cancer and pancreatic cancer.

7-12. (canceled)

13. The method of claim 1, wherein the hormone is selected from the group consisting of glucocorticoids, mineralocorticoids, androgens, estrogens, progestagens, thyroid hormone, melatonin, and leptin.

14. The method of claim 1, wherein the hormone is progesterone, estradiol, testosterone, or dehydroepiandrosterone (DHEA).

15-23. (canceled)

24. The method of claim 1, wherein the dynamic hormone index is the co-efficient of hormone variation.

25. The method of claim 1, wherein the subject is at risk if the dynamic hormone index of the subject varies by at least 25% from the mean or median dynamic hormone index of the population.

26. (canceled)

27. The method of claim 1, wherein the dynamic hormone index is determined by measuring the level of at least one hormone in two or more biological samples obtained from the subject at two or more time points.

28. The method of claim 1, wherein the dynamic hormone index is determined by measuring the level of two or more hormones in two or more biological samples obtained from the subject at two or more time points.

29. The method of claim 28, wherein the dynamic hormone index is determined by measuring the ratio of two or more hormones in two or more biological samples obtained from the subject at two or more time points.

30. (canceled)

31. The method of claim 27, 28 or 29, wherein the biological sample is a body fluid.

32-34. (canceled)

35. The method of claim 27, wherein the two or more biological samples are obtained at time points corresponding to the normal temporal pattern for the hormone.

36-40. (canceled)

41. (not entered)

42. The method of claim 1, wherein the population comprises subjects of with similar age, sex, socioeconomic level, race, ethnicity, body mass index, physical or other shared lifestyle variable as the subject.

43. A method of identifying a modifiable risk factor that has a statistical meaningful relationship with a hormone-associated condition comprising,
determining the dynamic hormone index of at least one hormone for individual subjects of a population, and
determining the level of exposure for the individual subjects of the population to a suspected modifiable risk factor,
wherein a meaningful statistical relationship between an aberrant dynamic hormone index of subset of the subjects in the population and the level of exposure of the subset to the risk factor is indicative that exposure to the risk factor increases the likelihood of developing a hormone-associated condition.

44. (canceled)

45. A method of determining whether there is a statistical relationship between a known risk factor and an aberrant dynamic hormone index comprising,
determining the dynamic hormone index of at least one hormone for individual subjects of a population, and
determining the level of exposure for the individual subjects of the population to the risk factor,
wherein a statistically meaningful relationship between an aberrant dynamic hormone index of a subset of the subjects in the population and the level of exposure of the subset to the risk factor is indicative that there is a relationship between the level of exposure to the risk factor and the dynamic hormone index.

46-66. (canceled)

67. A method of determining whether a subject has an increased risk of developing at least one symptom or adverse side effect of a hormone-associated condition comprising,
determining the relative level of at least one predictive hormone in two or more biological samples from the subject,
determining a dynamic hormone index for the subject,
wherein a dynamic hormone index for the subject that is aberrant when compared to the statistical distribution of the dynamic hormone index of the population is indicative that the subject has an increased risk of developing at least one symptom or adverse side effect of the hormone-associated condition.

68. (canceled)

69. A method of determining the level of risk for developing a hormone-associated condition in a subject comprising,
determining the level of at least one hormone in two or more biological samples at two or more time points from the subject; and
determining a dynamic hormone index for the subject, wherein the extent to which the dynamic hormone index of the subject varies from the mean or median dynamic hormone index for a population is indicative of the level of risk for developing the hormone-associated condition in the subject.

70. A kit for determining whether a subject has an increased risk of developing a hormone-associated condition comprising,
means for determining the relative level of at least one predictive hormone in at least one sample from the subject, and instructions for identifying whether the individual is at risk of developing the hormone-associated condition.

71-72. (canceled)
